(19) United States
(12) Patent Application Publication
(10) Pub. No.: US 2008/0306363 A1
(43) Pub. Date: Dec. 11, 2008

(54) Specialized Human Servo Device And Process For Tissue Modulation Of Human Fingerprints

(75) Inventors: Joseph Chaiken, Fayetteville, NY (US); Kylee Rudd, Dewitt, NY (US); Ethan Voss, Syracuse, NY (US); Rebecca J. Bussjager, Utica, NY (US); David Rice, Syracuse, NY (US); Daniel Godici, Brewerton, NY (US)

Correspondence Address: KAREN S. CANADY;CANADY & LORTZ LLP, COMMERCE PLAZA, 11340 WEST OLYMPIC BLVD., SUITE 275, LOS ANGELES, CA, 90064, US

(73) Assignee: Lightouch Medical, Inc., Bryn Athyn, PA (US)

(21) Appl. No.: 11/722,155

(22) PCT Filed: Jan. 6, 2006
(86) PCT No.: PCT/US2006/000409

§ 371 Date: Mar. 18, 2008

Related U.S. Application Data
(60) Provisional application No. 60/641,876, filed on Jan. 6, 2005.

(51) Int. Cl. A61B 5/1455

(52) U.S. Cl. 600/310

(57) ABSTRACT

Apparatus and methods for noninvasive spectroscopic measurement of an analyte in a subject that have been optimized for producing uniform and repeatable tissue modulation across test subjects and for the same test subject on different occasions are provided. The apparatus comprises an ergonomically shaped grip that substantially conforms to a subject's hand; a surface for placement of at least one of the subject's fingertips upon grasping the grip; and an optically transparent aperture, or a plurality of apertures, disposed within the surface. A modification to the surface of the apparatus adjacent to the aperture that is detectable via the tactile sense of the subject can be added to provide tactile feedback to the subject to guide correct placement of the fingertip over the aperture. The apparatus and methods can also incorporate feedback methods to guide and optimize placement and conditions of the fingertip to further improve accuracy of measurements.

This application claims the benefit of U.S. provisional application No. 60/641,876, filed Jan. 6, 2005. This application is related to the following commonly owned United States patents and applications: U.S. Pat. No. 6,044,285, issued Match 28, 2000; U.S. Pat. No. 6,377,828, issued Apr. 23, 2002; U.S. Pat. No. 6,223,063, issued Apr. 24, 2001; U.S. Pat. No. 6,289,230, issued Sep. 11, 2001; U.S. Pat. No. 6,292,286, issued Sep. 18, 2001; and Ser. No. 10/332,748, filed Jan. 13, 2003, and titled, “Method of Tissue Modulation For Noninvasive Measurement of An Analyte”, now U.S. Pat. No. ______. The entire contents of each of these patents and applications is incorporated herein by reference.

Throughout this application various publications are referenced. The disclosures of these publications in their entireties are hereby incorporated by reference into this application in order to more fully describe the state of the art to which this invention pertains.

### BACKGROUND OF THE INVENTION

Tissue modulation is the use of spatiotemporally localized mechanical, thermal, chemical and/or other external influences to manipulate the mobile components of tissue relative to the static components. Tissue modulation allows the use of difference spectroscopy to isolate the spectra of the mobile and static tissues. Modulation (spatial or temporal) is a crucial part of noninvasive spectroscopic analysis of human tissues and other living things in vivo. When the signal level for a particular type of spectroscopy is large, there is a greater likelihood of finding a passive approach to modulation that improves the quality, accuracy and precision of the resulting in vivo measurement. When the signal to noise ratio of the basic measurement process is too low, a more active approach, i.e. tissue modulation, can afford a substantial improvement over any non-modulated measurement approach.

It is instructive to consider the case of pulse oximetry in which visible, electronic state, absorption spectroscopy is combined with the natural fluctuation of the blood content of a particular part of the circulatory system, e.g. the fingertip pulse. The pulse provides an approximately 1-2 seconds per modulation cycle, and a ≈5% variation in local blood volume, and so sufficient signal to noise in the basic measurement, in this case the absorption of hemoglobin, must exist on that time scale or it cannot be used to obtain information about the substance being modulated, i.e. blood. If the signal to noise ratio, at least about 6:1, corresponds to a basic uncertainty greater than ≈5% then the modulation is insufficient. In the case of Raman spectroscopy, the signal to noise levels are much smaller and one has less flexibility in what will suffice for modulation. Fluorescence from electronically excited hemoglobin is a large enough signal for passive modulation and so we often can observe the pulse in the temporally resolved integrated fluorescence. For the usual signal levels obtainable today, the counting rate for Raman photons is not large enough to allow a 1-2 sec measurement cycle based on the blood volume modulation afforded by the pulse. Thus, tissue modulation is a necessity in Raman spectroscopy of blood in vivo. We recognize that, in this case, the basic tissue modulation process envisions subtracting two spectra where, as much as possible, the only difference between the two spectra is the blood content of the capillary bed.

Failure to involve modulation when it is necessary is the difference between success and failure. Signal levels themselves are not the only determinant. There have been numerous attempts to utilize near infrared absorption as a noninvasive approach to in vivo spectroscopic analysis of blood and other tissues (see Mark Arnold and Gerald Small, Futrex, Bico, Instrumentation Metrics and others). These have failed to a greater and lesser extent both because the spectroscopic probing was not able to locate features that were unambiguously associated with specific analytes or tissues and because the technique employed did not anticipate the natural fluctuations in such analytes and tissues in the volume being probed. That there are two factors precludes the success of “sufficient” modulation based on a single parameter. In some sense external modulation must be greater than natural fluctuations or it usually will not be possible to discern the natural fluctuations from the external modulation. When there are multiple sources of natural fluctuation, as just mentioned, multiple types of modulation are required. Failing to properly anticipate such variation, i.e. “natural” tissue modulation, the near infrared absorption technique cannot yield information about analytes at low enough concentrations to be clinically useful. Thus, near infrared absorption in vivo analysis of blood based on either forearm or tongue measurements has never provided much useful information at concentrations much below 200 mg/dl. Since normal glucose levels are 79-129 mg/dl, such measurements are of extremely limited value.

Generally speaking, the practitioners attempting to use these other types of spectroscopy have failed to employ techniques of tissue modulation or even to account for the normal fluctuations of blood and other tissue content during the course of their measurements. In the context of these attempts, fixtures have been developed (in particular see Arnold & Small and Instrumentation Metrics work referenced above) to hold the tongue or forearm area more or less motionless during the measurement process. This approach has been insufficient to deal with the overall problem. The overall problem is to insure not only that the tissue volume being probed is undergoing some type of appropriate tissue modulation, and enough different types of modulation, but that it is also being presented to an optical system in such a manner as to result in accurate and precise spectroscopic signals. In this respect, human factors cannot be overlooked when attempting noninvasive spectroscopic analysis of human tissues (and other living things) in vivo.

### SUMMARY OF THE INVENTION

The invention provides an apparatus and associated process for tissue modulation (e.g., of fingertip capillary beds) in the context of fluorescence and Raman spectroscopy. The process, devices and principles described have general utility and it will be apparent to those skilled in the art how to apply the approach to other tissues, how to adapt it to spatial modulation rather than temporal modulation, and to other types of spectroscopy.

The invention provides an apparatus for noninvasive spectroscopic measurement of an analyte in a subject. The apparatus comprises an ergonomically shaped grip that substantially conforms to a subject's hand; a surface for placement of at least one of the subject's fingertips upon grasping the grip; and an optically transparent aperture, or a plurality of apertures, disposed within the surface. In some embodiments, the apparatus further comprises a modification to the surface of the apparatus adjacent to the aperture. The modification is detectable via the tactile sense of the subject, and typically comprises at least one raised nub, bump and/or ridge on the surface of the grip. These modifications can be made of metal, plastic, rubber, glass, or other material. These modifications provide tactile feedback to the subject to guide correct placement of the fingertip over the aperture.

The shape of the grip itself can be modified to facilitate optimal placement of the subject's hand for both comfort and accuracy of data collection. The ergonomically shaped grip optionally comprises two or more ridges that define recesses, whereby the recesses conform to the subject's fingers upon grasping the grip. In another embodiment, the grip is shaped to facilitate placement of the volar side of the subject's thumb tip on a portion of the grip that opposes the surface for placement of the subject's fingertips, whereby the subject moves the fingertips and opposing thumb tip towards each other upon grasping the grip. This encourages a gripping motion that results in greater comfort and more effective application of force by the subject as compared to a motion that employs force using the wrist, forearm or upper arm of the subject.

Sensors can additionally be incorporated into the apparatus design to provide information regarding force applied by the subject's fingertip, proper positioning of the fingertip, temperature, and/or moisture or other conditions that can influence accuracy of measurements obtained via use of the apparatus.

In one embodiment, the apparatus further comprises a sensor in the surface adjacent to the aperture, wherein the sensor detects proper positioning of a fingertip over the aperture. Typically, the sensor comprises an electrode array. The electrode array can comprise a plurality of electrodes, each electrode detecting 60 Hz electrical activity. In one embodiment, fingertip position is detected using a set of one or more conductive spots and an annulus encircling the aperture so that electrical resistance between the spots and the annulus is monitored. The placement of the spots relative to the aperture is selected so that, if electrical resistance is below a certain level, the volar side of the fingertip must be in the correct location.

In another embodiment, the sensor detects pressure applied by a fingertip. Typically, the pressure-detecting sensor measures the applied force (preferably to within at least ±0.1 g precision on 0.02 sec timescale) with virtually no motion (e.g., <25 μm for ≈200 grams of applied force) of the aperture.

In yet another embodiment, the sensor detects moisture, e.g., by measuring resistance, capacitance or impedance between the electrodes. For example, measuring the moisture content (or dryness) of the fingertip skin can be used to ensure that the moisture content is amenable to optical spectroscopy without untoward artifacts. This can be determined by electrical resistance that is lower than a first pre-set level and greater than a second pre-set level. The same sensors that are used for moisture assessment can also be used for position sensing, or separate sets of electrodes can be used. The moisture and position monitoring can use Kapton® (Dupont), Mylar® (Dupont) or other nonconductive base material in a conventional flexible circuit board arrangement.

In some embodiments, the sensor detects temperature of the surface adjacent to the aperture and, optionally, the apparatus further comprises means for adjusting the temperature of the surface. Maintaining a particular temperature at the surface maintains mechanical stresses in the modulating surfaces (e.g., spring steel) and can be used to maintain a state of arterio-venous shunt for the purpose of thermal tissue modulation.

Optionally, the apparatus further comprises a feedback loop that transmits a detectable signal that corresponds to information detected by the sensor(s). The detectable signal can be transmitted to a processor and/or directly fed back to the subject via a display. Providing feedback to the subject and/or to associated automatic electromechanical apparatus can insure accurate and reproducible self-actuated tissue modulation or accurate and reproducible automatic tissue modulation. The feedback can be in a variety of forms, including, but not limited to, lights and/or sounds.

Typically, the apparatus further comprises a spectroscopic measurement system that directs light through the aperture toward the subject's fingertip and detects spectra emitted from the subject's fingertip through the aperture. The spectra to be detected and analyzed can be fluorescent, Raman and/or other spectra. Examples of other spectra include, but are not limited to, NMR, ESR, UV visible absorption, IR absorption, and phosphorescence spectra.

The invention additionally provides a method for noninvasive spectroscopic measurement of an analyte in a subject. The method comprises contacting the subject's hand with an apparatus of the invention and positioning a fingertip of the subject over the aperture. The method further comprises directing light through the aperture toward the subject's fingertip and collecting and measuring spectra emitted from the subject's fingertip through the aperture, wherein the spectra correspond to the analyte to be measured.

The method optionally further comprises monitoring the pressure applied by the fingertip positioned over the aperture and providing concurrent feedback to the subject regarding the pressure applied. The feedback can, for example, direct the subject to maintain application of a predetermined force, or to maintain application of a predetermined force per unit area of the volar side of the fingertip. The monitoring can be used to achieve “absolute isobaric” modulation. For example, each test subject of a plurality of subjects is directed to apply the same total force via either self-actuated or automatic tissue modulation. In this case, there will be a different amount of filtering (i.e. skim pool formation) for different subjects, whether pulse or tissue modulation is employed. This variation in apparent blood volume can be accounted for via algorithm. Alternatively, the monitoring can be used to achieve “differential isobaric” modulation. For example, each subject is directed to apply finger area scaled total force in either self or automatic actuated tissue modulation. In this case, there will be a nearly constant amount of filtering, i.e. “skim pool formation”, for different subjects, whether pulse or tissue modulation is employed. Constant “skim pool formation” means that the spectra will originate with samples having very nearly the same hematocrit and thus blood volume normalization by algorithm is simplified. This results in a less serious variation in apparent blood volume to be accounted for via algorithm. Finger area scaled force refers to width times length of volar side of distal segment of tissue modulated fingertip. This results in the same pressure (force per unit area) being applied for tissue modulation. In this case, variation from subject to subject in filtering is due to changes in blood pressure across subjects.

Information relevant to pressure monitoring can be found in H. Harry Asada and Stephan Mascaro, “Fingernail Sensors for Measurement of Fingertip Touch Forces and Finger Posture”, Progress Report No. 2-5, Mar. 31, 2000, http://darbelofflab.mit.edu/ProgressReports/HomeAutomation/Report2-5/Chapter08.pdf and references therein. Information relevant to thermoregulation can be found in http://hetkules.oulu.fi/isbn9514259882/html/c287.html.

In some embodiments, the method further comprises monitoring the position, electrical resistance or moisture of the fingertip positioned over the aperture and providing concurrent feedback to the subject regarding the position, electrical resistance or moisture of the fingertip. In some embodiments, the method further comprises rejecting a spectral measurement if the resistance of the fingertip does not fall within a predetermined range. These monitored features can be stored in association with the concurrently collected spectral data. Should it be determined that data collected under conditions meeting certain criteria, e.g., at risk of resulting in inaccurate spectral measurements, the data collected under those conditions can be discarded.

Also provided are a method, system and an article of manufacture, referred to as a “PDPM”, comprising a program storage device readable by a computer and tangibly embodying one or more programs of instructions executable by the computer to perform method steps for monitoring the position, electrical resistance or moisture of the fingertip positioned over the aperture and providing concurrent feedback to the subject regarding the position, electrical resistance or moisture of the fingertip. The segment of the LighTouch® system that is relevant here comprises a computer having a display device and an input device attached thereto, and an application program, executed by the computer, for receiving commands from a user via the input device and for generating an output command stream in response thereto, wherein the output command stream comprises one or more instructions for directing the method set forth in the flow chart of FIG. 30 and to be executed by the PDPM.

This flowchart assumes the existence of a master program resident in the computer that in preparation for a measurement cycle/experiment sends the PDPM initialization parameters, e.g. total length of measurement cycle in time and/or CCD frames, duration of a single CCD frame, target pressures for each part of the tissue modulation cycle, target resistances including tolerances designed to trigger main feedback software to alert test subject/LighTouch® operator that targets were not being achieved, what location to send data to after the experiment cycle and other parameters. By monitoring a communication line for an “initiate measurement/experiment cycle” signal in concert with a “status heartbeat”, i.e. an electrical clock-like signal that the master computer uses to provide temporal synchronization to the various subsystems, the PDPM initiates a measurement/experiment cycle or performs the tasks needed because one has already been initiated and/or is in progress. If an experiment/measurement cycle is not in progress, then the PDPM monitors the communication lines for a “heartbeat” and initialization signals from the master. If a cycle is in progress then the PDPM must read all the sensors, buffer the data, compare readings with preset levels, consistent with values inserted during initialization and then update light emitting diodes (LEDs) to thereby provide needed teal time feedback to test subject. Comparing elapsed time (from counting heartbeats) and/or number of CCD frames collected, the PDPM either continues experiment or recognizes that the measurement cycle is complete.

At the end of a measurement cycle (i.e. tissue modulation cycle), the data associated with these transducers can be transferred to the master computer to be stored along with the CCD camera data. Once transferred the computer system can examine the transducer data and the camera data, apply data integrity screens, sort the data, and then allow glucose and other analyte concentrations to be calculated based on the valid part of the overall data that were collected.

### BRIEF DESCRIPTION OF THE FIGURES

FIG. 1: Raw pressed (black-inset, lower line) and un-pressed (red-inset, upper line of inset) spectrum and difference spectra (uppermost line) across range ≈350-≈1750 cm−1.

FIG. 2: Raw difference spectrum (blue) from ≈350 cm−1 to ≈600 cm−1 also showing baseline (red) used for integration. Also shown in black is an appropriately scaled spectrum of =1800 mg/dl glucose in gelatin that allows observation of relevant glucose features.

FIG. 3: Clarke Error Grid analysis of 49 data points. Zone labels are as described in text. The 18 one individual calibration points are shown in black. The 8 same patient validation points are shown in red. The data points from random patients are shown in green triangles except for the ones we shall label and number as “random individual-outliers”. The various colors and shapes are maintained consistently in subsequent relevant figures to allow tracking of these points throughout the disclosure.

FIG. 4: HemoCue® glucose plotted as a function of HemoCue® hemoglobin. Linear regression gives r=0.16, SD=50. N=49, p=0.92.

FIG. 5: LighTouch® glucose plotted as a function of HemoCue® hemoglobin. With all points included (inset) linear regression gives r=−0.42, SD=48, N=46, p=0.004 and with one (E zone) outlier removed r=−0.27, SD=39.1, N=45, p=0.073.

FIG. 6: The difference between HemoCue® and LighTouch® glucose measurements plotted as a function of HemoCue® hemoglobin. With all points included as shown linear regression gives r=0.38, SD=56.0, N=46, p=0.009 and, with one (E zone) outlier removed, r=−0.19, SD=41.2, N=45, p=0.212.

FIG. 7: The difference between HemoCue® and LighTouch® glucose measurements plotted as a function of total modulated fluorescence counts. With all points included linear regression gives r=0.000, SD=58.7, N=49, p=0.97 and with one outlier (E zone) removed r=−0.19, SD=41.2, N=48, p=0.21.

FIG. 8: The modulated fluorescence counts plotted as a function of hemoglobin concentration. With all points included linear regression gives r=−0.07, SD=1.45E8, N=45, p=0.64 and with four highest modulation outliers removed r=0.25, SD=7.47E7, N=41, p=0.106. Black line connects five of six “outliers” based on Clarke Error Grid analysis (FIG. 3) forming “boundary” for rejecting data as described in text.

FIG. 9: TA.XT Texture Analyzer experiment simulating tissue modulation of fingertip showing force variation with amount of pressing, i.e. displacement or distance squeezed from testing state and then during release of pressing.

FIG. 10: The difference between HemoCue® and LighTouch® glucose measurements plotted as a function of the average of the HemoCue® and LighTouch® glucose measurements. With all points included (inset) linear regression gives r=−0.07, SD=58.5, N=49, p=0.644 and with one outlier removed r=0.23, SD=39.7, N=48, p=0.12.

FIG. 11: LighTouch® glucose plotted as a function of HemoCue® glucose. With data selected as described in text r=0.80, SD=22, N=38, p<0.0001 and with three highest points excluded r=0.70, SD=21, N=36, p<0.0001.

FIG. 12: Digital image of a human subject's hand gripping one embodiment of the grip of the invention. A molded grip has ridges to guide placement of the fingers.

FIG. 13: Digital image of additional embodiments of the grip of the invention. The various views show how the grip is adapted to communicate with a LighTouch® spectroscopic measurement device (via window, hollow center and support plate).

FIG. 14: Graph showing the measured resistance between two electrodes connected by a finger placed over the electrodes. The finger was either dry, i.e. without added water, moistened and then dried with a paper towel or mildly lotioned with all excess being toweled off.

FIG. 15: Digital image of an embodiment of the grip that incorporates a nonflexible digital signal processing board. The printed circuit board shown in this image incorporates four electrodes surrounding a central hole that serves as the tissue modulation aperture. A pressure sensor is shown, with its coaxial cable coming off to the left.

FIG. 16: Digital image of the embodiment shown in FIG. 15, with a subject's hand and fingers in place on the grip.

FIG. 17: Image of a Feedback Window produced by the reporting software of the invention. Four spots represent the electrode pattern of the processing board shown in FIG. 15. Each spot will appear green when the corresponding electrode is properly contacted, and red if it is not. Numerical values associated with each spot indicate moisture content, permitting evaluation as to whether moisture content is within and acceptable range of values. Near the top of the window is an indication of pressure units and a comparison target pressure value. In this embodiment, the pressure units are calibrated in absolute grams. The pressure is indicated here with both a numerical readout and a sliding scale to assist the user in adjusting pressure to bring the actual pressure into registration with the target level. The pressure target, set in the master program before a measurement cycle is user initiated, can change as the tissue modulation cycle changes, with guidance provided in this feedback window for the patient to adjust accordingly.

FIG. 18: Image of a computer window displaying blood volume over time (“wave number” in this graph refers to frame number).

FIG. 19: Image of a computer window displaying real-time information from a 20-second test.

FIG. 20: Close-up image of the same three panes displayed in FIG. 19, expanding the view of the test subject involuntary “flinch” at frame 517.

FIG. 21: Image of computer window displaying tissue modulated spectrum obtained from a 200 second tissue modulation cycle, with baseline removal and corresponding spectral features from a glucose containing calibrator spectrum (lower spectrum).

FIG. 22: Schematic diagram showing an oblique view and a top view of an idealized grip.

FIG. 23: Digital image of subject's hand and fingers engaged with fingertip over aperture of apparatus.

FIG. 24: Digital image of top view of subject's hand engaged around grip and with fingertip over aperture of apparatus.

FIG. 25: Digital image close-up of aperture surrounded by annular and other electrodes 250. Nubs 252 are positioned above and below the annular electrode 250. Also shown are the Kapton® flexible circuit board 254 and the cutaway 256 from the spring steel to adjust tension.

FIG. 26A (side view) and FIG. 26B (front view): Detailed schematic of tissue modulation surface of apparatus showing lever arrangement and placement of transducer(s) relative to fulcrum in a single force transducer version. Depicted are a load cell/strain gauge (LCKD type, Omega Engineering Inc., Stamford, Conn.) 260, conduit for electrical connections 261, base plate 262, cut-outs to adjust spring steel tension 263, spring steel 264, thermistor or thermocouple conduit 265, orifice or aperture for probing light to contact tissue 266, and fulcrum 267.

FIG. 27A (side view) and FIG. 27B (front view): Detailed schematic of tissue modulation surface as in FIG. 26A-B, but in a two force transducer version. Depicted are multiple transducers to detect sheet modulation 270, base plate 262, spring steel 264, orifice or aperture for probing light to contact tissue 266, and fulcrum 267. “Sheet modulation” means the test subject did not apply force directly perpendicular to the modulation surface. Instead, the subject applied a significant force component parallel to the modulation surface, which leads to peculiar results.

FIG. 28: Graphic display of pulse shape (net amount of blood passing a certain point in a fingertip) plotted as blood volume (amplitude) over time (frame number) in a subject using the apparatus obtained with minimal applied force (force of fingertip against aperture).

FIG. 29: Graphic display of pulse shape (net amount of blood passing the same point as in FIG. 28) plotted as blood volume (amplitude) over time (frame number) in a subject using the apparatus obtained with increasing amounts of applied force (force of fingertip against aperture).

FIG. 30: Flow chart illustrating software function for sensor monitoring and feed back subsystem, i.e. PDPM.

FIG. 31: Display of spectra showing effect of systematically increasing pressed target force with constant un-pressed target force. As force during pressed part of cycle increases, the hematocrit in skim pool, i.e. red region that forms inside orifice, becomes less until it teaches a minimum, usually not zero.

FIG. 32: TA.XT traces showing effect of increasing applied force on pulses through capillary bed. Pulses are at first small because there is little mechanical coupling between force transducer and skin surface/vasculature. At intermediate applied force, the pulses become large and distinct until at highest applied force pulses become less well defined and more erratic as heart cannot pump blood past obstruction(s) created by applied force. Lowest forces at bottom trace with increasing force going upward.

FIG. 33: Raw pulse modulated Raman spectrum showing intermediate skimming effect.

FIG. 34: Raw pulse modulated Raman spectrum in FIG. 33 after background subtraction in which a 101 adjacent average is subtracted from the raw spectrum and the result is 7 point adjacent average smoothed.

FIG. 35: Fluorescence spectra of plasma with 785, 805 or 830 excitation.

FIG. 36: Emission spectra of plasma, serum or hematocrit spiked water or phosphate buffered saline with 785 nm excitation.

FIG. 37: Optically observed pulses obtained by integrating the total emission for >800 cm−1 Raman shift. The inset shows the same data between frames 2000-2200 (un-pressed) and frames 9000-9200 frames (pressed). Note external tissue modulation pressure was applied just past frame 5000. Slight variation in applied force near frame 4000 results in (involuntary) blood flow.

FIG. 38: First derivative of pressed part of FIG. 37 showing pressure waves.

### DETAILED DESCRIPTION OF THE INVENTION

The invention described herein substantially alleviates problems encountered with in vivo spectroscopy by providing a method and apparatus for producing uniform and repeatable tissue modulation across test subjects and for the same test subject on different occasions. Simply being motionless for a particular measurement time scale with or without tissue modulation is an obvious requirement for any high quality in vivo measurement system. Anatomy, metabolism and physiology cannot be ignored in determining the necessary parameters for modulation. There are some factors that, if they cannot be controlled, they can at least be monitored and their effects on the measurement process anticipated.

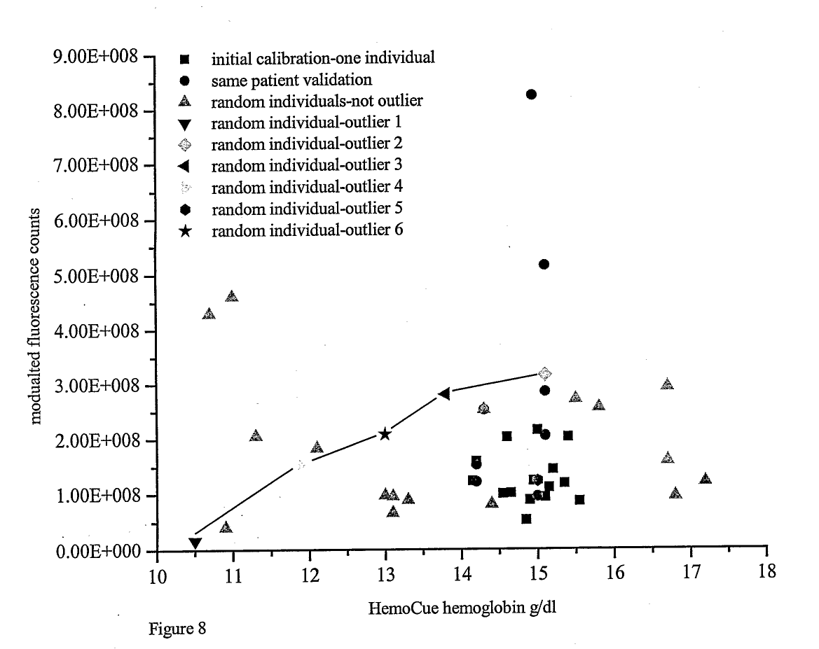

A particular set of measurements that illustrate the situation can be seen in FIG. 8, which shows the amount of tissue-modulated fluorescence plotted as a function of the independently measured (HemoCue® fingerstick; Ängelholm, Sweden) hemoglobin concentration. In this case, the test subject, pushing his or her fingertip against an aperture in a rigid metal plate, executes the tissue modulation. Exciting light impinges on the capillary bed in the fingertip through the hole and the fluorescence from the hemoglobin in the blood is monitored. By regarding a section of the circulatory system as a set of tubes filled with fluid, one would expect, based on Poiseville's equation and related principles, a smooth increasing variation of the tissue modulated fluorescence with increasing hemoglobin concentration. That there are any significant outliers, such as deviations greater than about 2 standard deviations from the mean, in FIG. 8, is a function of either circulatory pathologies or improperly executed tissue modulation. Minimizing the occurrence of such outliers is one object of the present invention.

Neuropathy can itself be a cause of improperly executed tissue modulation. There is undoubtedly a greater tendency toward improperly executed tissue modulation when there are circulatory issues that result in neuropathies impairing the test subject's capacity to detect on their own, improperly executed tissue modulation. Neuropathy impairs test subject's tactile response. Even including variation from person to person in the density of capillaries, size of capillaries or average erythrocyte diameter, we see a number of outliers (inset) that do not belong to the obvious general trend. As expected, the general trend is in actual fact for the amount of tissue modulated fluorescence to increase with the hemoglobin concentration as can be seen in the plot after neglecting the outliers.

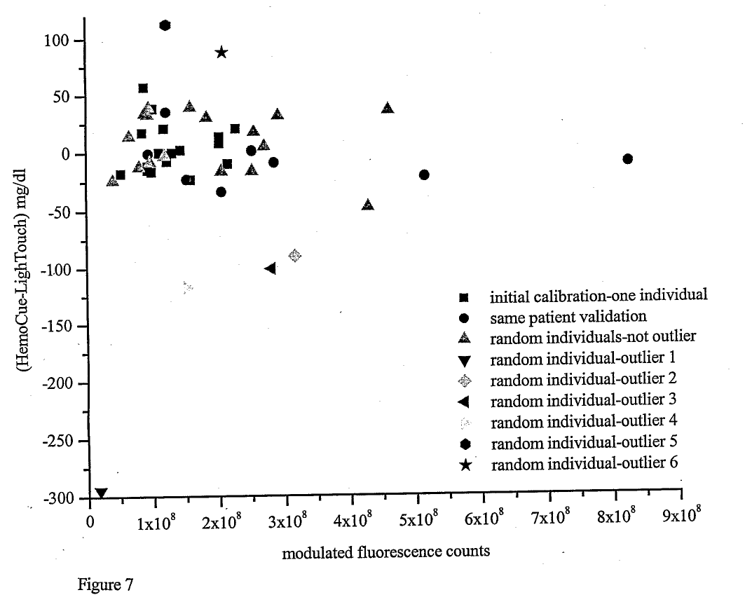

That such outlets can lead to spurious analysis of blood can be seen in FIG. 7. The apparent error in a LighTouch® noninvasive spectroscopic measurement of blood glucose relative to a HemoCue® fingerstick (using drawn blood and conventional glucose detection) measurement is plotted as a function of the tissue modulated fluorescence induced by laser excitation at 785 nm in finger tip capillary beds. (See U.S. Pat. No. 6,289,230, issued Sep. 11, 2001.) The modulated fluorescence is a measure of the amount of hemoglobin (i.e. proportional to blood volume) modulated from the spectroscopically probed volume (in this case) under the influence of mechanical tissue modulation, i.e. pressure applied to region to produce a relative depletion of the local capillary bed. There is a clump of measurements corresponding to roughly 1-3 E8 counts for which the tendency is to produce an acceptable deviation. The exact value of the number of counts relates to the specific experimental conditions being employed, but the number is a function of the laser power and wavelength, collection/excitation system optics, detector efficiency, capillary density, hematocrit/hemoglobin concentration, oxygenation, carboxyhemoglobin concentration, poisoned hemoglobin due to tobacco smoking and other factors. The tendency to produce an acceptable deviation is also a function of the fact that HemoCue® has finite accuracy and precision.

To fully appreciate the information contained in the figures, consider the relationship between a number of the outliers in FIG. 7 and the corresponding data in FIG. 8. Often but not always, a data pair in FIG. 7 which appears to be an outlier corresponds in FIG. 8 to a data pair for which a large modulation occurred for a person with a low hemoglobin concentration. Similar correspondences also occur, but in which a low modulation corresponds to a high hemoglobin concentration. There are anatomical differences between persons that could account for this observation in certain occasions. Persons with large fingers tend to have more blood to modulate. But one explanation follows directly from observations made at the time of measurement. Observing the impression made in the fingertip due to the pressure employed during the tissue modulation cycle indicates that not all test subjects employed the same pressure and also that not all test subjects probed the same location on the fingertip. Also, conversations with the test subjects have revealed that they often cannot feel the hole during any portion of the tissue modulation cycle so sometimes the finger may not have been in registration with the aperture at all.

This was known before the data were collected for FIGS. 7, 8 and below. To alleviate this problem, we employed a set of nubs positioned around the tissue modulation aperture that the test subject could feel and therefore employ to insure that the finger was positioned correctly. The relevant human factors involve:
1) incorrect fingertip placement relative to the tissue modulation aperture,
2) lack of uniformity in amount and direction of applied pressure during the various cycles of tissue modulation,
3) lack of uniformity in the skin optical properties that cannot be avoided by tissue modulation because of the inability of some test subjects to hold their fingers motionless relative to the position of the tissue modulation aperture during the course of tissue modulation therefore precluding successful subtraction of pressed from unpressed spectra,
4) all of these factors must be dealt with simultaneously and in real time to produce products that employ tissue modulation.

The invention disclosed herein solves these and other problems.

### DEFINITIONS

All scientific and technical terms used in this application have meanings commonly used in the art unless otherwise specified. As used in this application, the following words or phrases have the meanings specified.

As used herein, “Mie limit” refers to electromagnetic radiation interacting with materials having a characteristic size about equal to the wavelength of the electromagnetic radiation. Mie limit scattering typically occurs in the presence of scattering bodies that are approximately 50% of the size of an incident laser wavelength.

As used herein, “aperture” refers to an opening in a device through which light passes. The opening can be a physical opening, such as a hole in the device, or it can be merely an area that is sufficiently transparent to allow light to pass through. The aperture permits the direction of light onto a target or sample to be probed. A structure that, in conjunction with the skin, can produce a stress field in the tissues such that there is a flow of blood, or interruption of flow of blood, while simultaneously allowing the tissue to be contacted with light is an aperture.

As used herein, “tissue” means any portion of an organ or system of the body, including, but not limited to, skin, capillary beds, blood, muscle, breast and brain.

As used herein, “Raman spectra associated with” a given component refers to those emitted Raman spectra that one skilled in the art would attribute to that component. One can determine which Raman spectra are attributable to a given component by irradiating that component in a relatively pure form, and collecting and analyzing the Raman spectra emitted by the component in the relative absence of other components.

As used herein, “tissue modulation” refers to the modulation of blood flow and/or content within a target tissue. The modulation achieves blood replete and blood depleted states within the target tissue.

As used herein, “blood replete” refers to a state in which blood flow through a tissue is unobstructed by, for example, vasoconstriction induced by cooling or the application of pressure. The blood replete state can be enhanced by conditions that increase vasodilation, or modulation of the arteriovenous shunt, such as warming. It can also be enhanced by chemicals that increase or decrease blood flow such as capsicum, histamines, or other chemicals.

As used herein, “blood depleted” refers to a state in which blood flow or content through or in a tissue is substantially restricted and blood volume is minimized. A blood depleted state can be achieved by, for example, cooling and/or applying pressure to the tissue.

As used herein, “portion of tissue” refers to an area of tissue that light penetrates, and from which a signal is collected. A “target tissue” refers to an area of tissue that is to be probed for signal collection.

To address the lack of uniformity in spectroscopic probing and tissue modulation mentioned above, the present invention provides the following:

(1) The use of nubs (similar to Braille dots) to give real-time feedback to the test subject concerning the positioning of the fingertip and the amount of pressure being employed. Other measures can be employed that would give more precise and detailed information. The invention provides a measure independent of the honesty/competency of the test subject to assess compliance with the tissue modulation condition. For example, a “fixture” allowing for accurate placement and tissue modulation will be more successful when the design of the fixture is ergonomic and comfortable. Auditory cues (e.g. tones) and visual cues (e.g. via display on computer screen or other monitor) can also be given to the subject to provide feedback as to the sufficiency of pressure applied and/or to signal the subject when it is time to switch between a “pressed” and “unpressed” state.

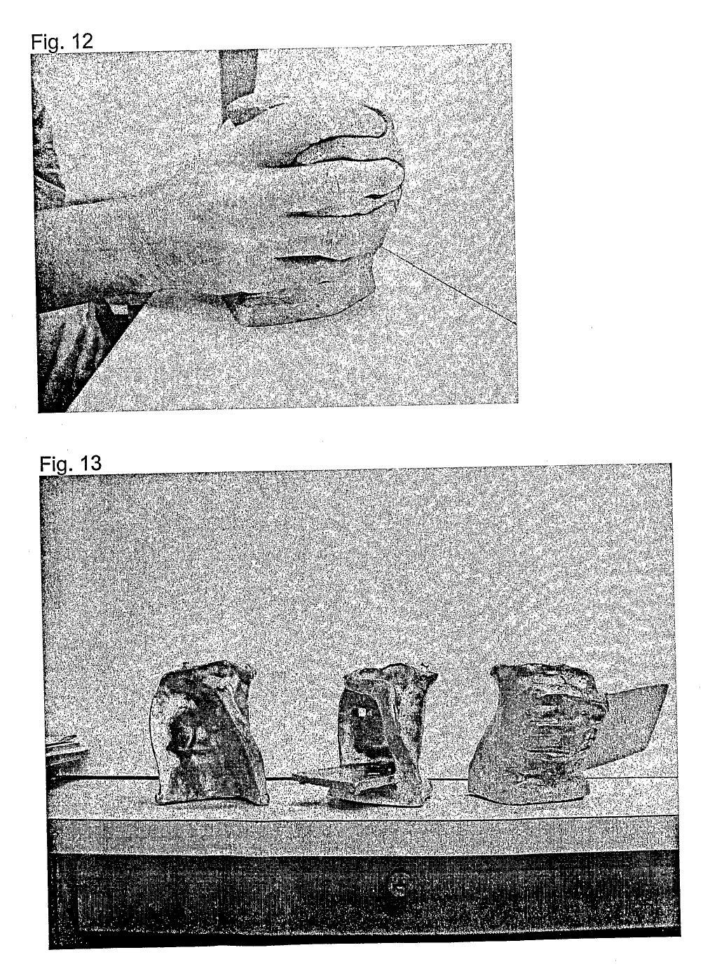

Thus (2) the shape of the fixture should be tuned to the shape of the tissue to be modulated. For use with fingertips, the invention provides a fixture such as the one shown in FIG. 12. Those in the art will appreciate that other shapes can be made that achieve the same objective. The desired shape resembles a specialized “grip”. This “Optical Grip™” allows for easy tissue modulation while also permitting easy access to the tissue by exciting laser radiation and a specialized light collection system. Further, because the hand and fingers are in a natural gripping motion and position, it is easier for anyone to maintain both positioning and pressure throughout any particular modulation cycle, as well as from one cycle to another cycle. In some embodiments, the grip will have raised ridges between the fingers (or recesses that create ridges between the fingers) that provide stability, comfort and confirmation of accurate placement to the hand. The shape preferably allows as much opposition between the fingers and thumb as possible. Opposition of fingers and thumb provides maximum comfort and ease of pressurization, while still giving as much access to the capillary beds as possible. In this respect, a more grazing angle is preferred to a normal angle of incidence between the exciting laser beam and the skin surface to insure that the radiation impacts as many capillaries side on as possible. At the same time, the optical axis of the light collection system is preferably along the normal to the skin surface, with the two optic axes (excitation and collection) to intersect at the capillary beds closest approach to the stratum corneum, i.e. about 200 microns below the skin surface. These two considerations are opposing, and so a particular grip design will embody a trade off between the two. In one embodiment, the grip is designed to permit measurement from more than one fingertip simultaneously. The shape and orientation of the grip should also take into consideration the disposition of the elbow and forearm, for comfort.

The position of the entire hand in the grip can be ascertained and corrected. To insure uniform placement of the finger relative to the aperture, the invention additionally provides (3) the use of an electrode array that the finger contacts when in proper registration with the aperture. By measuring the DC or AC electrical impedance, resistance and/or capacitance between the electrodes, one can be certain the skin is providing contact to each electrode.

Another way (4) to insure that each electrode is contacted, is to detect the 60 Hz pick-up on the skin by each electrode separately. The skin is well known to act as an antenna for leaked electromagnetic radiation from the ubiquitous 60 Hz AC power grid.

To insure that the proper pressure is applied in each of the tissue modulation cycles, no matter how many are utilized, the invention provides (5) the use of a pressure sensor (Omega LCKD series or similar; Stamford, Conn.) to measure in real time the actual pressure applied to the capillary bed in question.

Since the optical properties of the skin are, to a large extent, determined by moisture content, i.e. dry cracked skin scatters more and provides greater measurement sensitivity to small motions during the modulation cycle, the invention provides (6) a means to measure the resistance and/or capacitance and/or impedance between the same electrodes used for positioning to assess the degree of hydration before the overall measurement cycle begins. This latter technique can be adapted from the cosmetic industry and the food science industry and is performed at the beginning of a measurement cycle.

If the electrical resistance is too high or low compared to internally stored data, for example, then the LighTouch™ can (7) decline/defer/refuse to initiate a measurement cycle and suggest that the test subject use a moisturizer with the correct optical properties as well as moisturizing characteristics.

In the same vein all this information (8) can be monitored in real-time by an appropriately integrated software system that (9) interprets the data and (10) provides real-time instruction to the test subject, using various types of human-electronic interfaces, so as to cause the test subject to move his/her finger to the proper location, to press harder/softer, or to be more motionless. The software system sorts (11) the optical data as it is collected so as to exclude data collected during questionable periods of the overall measurement cycle. In this way only data collected during precisely controlled tissue modulation will be included in the analyte calculations.

### EXAMPLE 1

### Effect of Hemoglobin Concentration Variation on Glucose Analysis Using Tissue Modulated, noninvasive, In Vivo Raman Spectroscopy of Human Blood: a Small Clinical Study

In this Example, tissue modulated Raman spectroscopy was used noninvasively to measure blood glucose concentration in people with Type I and Type II diabetes with HemoCue® fingerstick measurements being used as reference. Including all of the 49 measurements, a Clarke Error Grid analysis of the noninvasive measurements showed that 72% were A range, i.e. clinically accurate, 20% were B range, i.e. clinically benign, with the remaining 8% of measurements being essentially erroneous, i.e. C, D, or E range. Rejection of 11 outliers gave a correlation coefficient of 0.80, a standard deviation of 22 mg/dl with p<0.0001 for N=38 and places all but one of the measurements in the A and B ranges. The distribution of deviations of the noninvasive glucose measurements from the fingerstick glucose measurements is consistent with the suggestion that there are at least two systematic components in addition to the random noise associated with shot noise, CCD spiking and human factors. One component is consistent with the known variation of fingerstick glucose concentration measurements from laboratory reference measurements made using plasma or whole blood. A weak but significant correlation between the deviations of noninvasive measurements from fingerstick glucose measurements and the test subject's hemoglobin concentration was also observed.

Studies show that intensive self-monitoring of blood glucose by people with diabetes can allow them to maintain blood glucose concentrations at near normal values. Improved glucose levels delay the onset and progression of long term consequences of poorly managed diabetes including, but not limited to, peripheral neuropathy, circulatory damage, retinopathy, and early death. The advent of commercial fingerstick devices allowing self-monitoring in the late 1970s represented a landmark in diabetes care. These observations stimulated an effort to discover a totally noninvasive method. In this section, we describe the results of a pilot clinical study to evaluate the accuracy and precision of a noninvasive technique based on tissue modulated Raman spectroscopy.

Earlier studies describing noninvasive Raman spectra of human blood in vivo were designed to establish that the source of the spectra was indeed blood and that the spectra were volume normalized so that quantitative noninvasive blood analysis should be possible. Since the spectra contain features that are easily seen to be associated with many well-known biological materials, we found that glucose is an important and feasible target analyte. Raman spectroscopy has long been an important technique for analysis of mixtures at the millimolar concentration levels with or without the use of common chemometric methods and thus could be used for other analytes in blood.

In this section, we describe improvements in instrumentation and methodology as well as in blending human performance factors into the overall method. Although a technique might be quite useful as a research tool for trained scientists and lab technicians, the same technique must meet many requirements if it is to be applicable for the large scale self-monitoring of blood glucose by untrained persons. While there have been studies (e.g. near IR transmission and diffuse reflectance) showing how other noninvasive or minimally invasive techniques can be used to monitor blood glucose in the hyperglycemic range, any technique applicable for the large scale self-monitoring of blood glucose must provide accurate and precise measurements in the normal glucose concentration range (79-129 mg/dl). Although there is utility in having a non-portable device for clinical settings, an inexpensive portable device is necessary if it is to be used by patients throughout the day. The algorithm employed in this study was developed because it resembles the use of single channel detectors and filters as we suspect would be required for miniaturization. Blood and circulatory abnormalities such as abnormal hemoglobin concentrations and peripheral circulatory and nervous pathologies need to be addressed before a noninvasive blood glucose monitor can marketed to patients with diabetes.

### Experimental Apparatus and Procedures

In accordance with our Institutional Review Board (IRB) approved protocol, all subjects provided informed consent. Subjects included 23 males and 2 females with diabetes mellitus ranging in age from 21 to 70 years.

The experimental apparatus (LighTouch® noninvasive spectroscopic measurement device) produced a glucose determination that was compared to HemoCue® (HemoCue, Lake Forest, Calif.) glucose measurement, a validated device for capillary glucose. The hemoglobin was measured using the HemoCue® Hb device. The LighTouch® device was operated by a technician not having an advanced physical science or engineering degree. Data was archived by an independent party who also performed all fingerstick blood glucose and hemoglobin measurements. Different lots of HemoCue® (Lake Forest, Calif.) test cuvettes were mixed randomly throughout the study and whenever possible, i.e. with the discretion of the test subjects, fingerstick measurements were repeated and the results of the two fingersticks were averaged. On a single occasion two fingerstick measurements were made within minutes of each other and they differed by a large amount, i.e. 100 mg/dl. Although spiking is a known occurrence, a third measurement was attempted to be sure. The three measurements were compared and one measurement was eliminated on the basis of a Q test at 90% confidence. The fingerstick average was always paired with a single LighTouch® measurement that was performed within 3-4 minutes of the set of HemoCue® measurements. Previously described tissue modulated spectroscopy was used for the present study with certain modifications. The results obtained in this study were obtained using 31 mW at the sample, continuous wave, 785 nm wavelength excitation from an external cavity diode laser (Sachet Laser, Marburg, Germany) that is filtered by two laser line excitation clean-up filters purchased from Omega Optical (Brattleboro, Vt.). This laser power produced no sensation as demonstrated by the fact that no test subject was able to discern whether the laser was on or off. This power and focusing corresponds to less than the maximum permitted exposure (“Laser Safety”, Henderson and Schulmeister, Section 3.8, Institute of Physics Publishing, Bristol and Philadelphia, 2004) which is about 33 mW. Although going to longer wavelengths will permit an increase in the maximum permitted exposure as well as other benefits (e.g. reduced fluorescence), we note that we have obtained quite good results on the same overall timescale using 785 nm excitation with powers as low as 19 mW at the sample. The filtered beam was focused using a single fused silica lens to a nominal spot size of 100 μm diameter when the laser spot is observed using a flat surface inserted into the beam at normal incidence. This is a nominal spot diameter because in practice the laser impinges on the stratum corneum of the volar side of the distal segment of the middle finger of the test subject at an angle of 53 degrees. Therefore the actual spot shape on a non-scattering target is approximately elliptical with a major axis of at least 167 μm and a minor axis of about 100 μm.

Tissue modulation was accomplished using a 2.1 mm diameter hole in a 1 μm thick aluminum plate. The hole also serves as the aperture for the light and is beveled outward on the side opposite the surface that contacts the fingertip. Three small nubs arranged in a triangle around the hole on the side facing the fingertip have dimensions taken from standard Braille. These nubs allow test subjects to use their sense of touch to orient the location of their fingertip relative to the aperture. This hole/aperture size is smaller than earlier prototypes, allowing substantially less protrusion of stratum corneum into the aperture thereby providing both a more mechanically stable focal point for the optical system and a more uniform stress field to affect the tissue modulation process itself.

After a measurement cycle, the nubs and the hole produce temporary but observable indentations on the skin allowing the LighTouch® operator to assess the position of the measurement, to suggest to what degree the test subject was motionless during the measurement cycle, and to indicate how hard the test subject pushed against the tissue modulator during the pressed period. It is preferred that the aperture be motionless throughout the tissue modulation cycle. (In the particular embodiment described here, not much more than ≈35 microns of motion can be tolerated without degradation of performance.)

The light emanating from the irradiated zone is collected and collimated by a fused silica single lens before it is filtered by a holographic notch filter, (Kaiser Optical Systems, Ann Arbor, Mich.), and subsequently refocused by another lens onto the input side of a hexagonal packed, nearly circular profile, 59 fiber×100 μm, fiber bundle. The fiber bundle, (Process Instruments, Salt Lake, Utah), is configured to form a line image on the output side where it brings the light to a 1200 grooves/mm spectrograph (also Process Instruments, Salt Lake, Utah). The entire collection and dispersal system is approximately f=2.1. The spectrograph disperses the collected light onto a CCD camera (Andor Technologies, South Windsor, Conn.) having 256 vertical and 1024 horizontal pixels and is operated at −85° C.

In order to explore quantitatively the tissue modulation process, some experiments were performed with a TA.XT Texture Analyzer (Stable Micro Systems, Surrey, England). There are many ways to configure a TA.XT. In out study the TA.XT precisely moves a mechanical probe while simultaneously recording the force, displacement and time of the probe with respect to a fixed reference position and time. The force and displacement are both recorded with a bandwidth of 0.2 KHz, with the force accurate and precise to ±100 mg and the displacement resolution accurate and precise to ±10 microns. We used a probe of out own design that contacts the volar side of a fingertip with a flat aluminum surface having a 0.21 cm diameter hole such that the overall interaction of the probe with the fingertip is essentially identical to that of a fingertip making contact and pressing on the tissue modulator aperture. The finger itself is held at rest in a bed also of our own design that is machined out of aluminum. The bed has a half-cylindrical cross section and allows the test subject's hand and fingertip to test comfortably while the TA.XT brings the probe down onto the fingertip. In this way the fingertip remains motionless while the TA.XT probe moves.

A LighTouch® device data collection sequence for an uninitiated test subject begins with a short training session. Sitting in a dark room with only the laser and computer monitor turned on, the test subject is allowed to observe the wavelength-dispersed output of the Andor camera in teal time, i.e. a continuously updated sequence of 20 msec frames. Initially the test subject is requested to place only sufficient pressure against the aperture as is needed to insure that the skin is flush against the metal forming the aperture. While there are independent means to assure that this is the case, in this study the test subjects used their tactile powers. Typically this “unpressed stage” of a tissue modulation cycle corresponds to about 1 Newton total force. On a single frame basis, a spectrally broad fluorescence is always observed in addition to small but unmistakable Raman features corresponding to amide I and CH₂ deformation modes at about 1670 cm⁻¹ and 1450 cm⁻¹ Raman shift respectively. The test subject is then requested to push gently against the aperture while watching the real time response of the CCD camera. As long as the physical contact is not broken, any additional pressure, typically 2-7 Newtons depending on the relative size of the person's finger and the person's blood pressure, results in a relative emptying of the irradiated capillary bed. In this “pressed” state, all subjects were able to observe the fluorescence signal and Raman signal decrease in concert. Instructing the test subject to release some pressure, while maintaining continuous contact with the aperture, makes an equally obvious increase in the CCD response. The test subject is invited to press and release a few times while maintaining constant contact and observing the real-time response to gain experience with the “feel” of the process.

Observing these changes allows the test subject to calibrate their own hand as a “servo” unit with regard to producing either a “pressed” or “unpressed” state. In some cases we allowed the test subject to experience a short practice run in which he/she was instructed to produce an unpressed state for 10 sec and then a pressed state for 10 sec during which time he/she was not permitted to see the teal time CCD response because the computer monitor was intentionally turned off. The transition between states was initiated by audio cues from the software to the test subject. Afterwards the software produced a graphical representation of the blood volume versus time profile by plotting the integral of the fluorescence (see below) as a function of frame number, i.e. time. This representation allows the test subject and LighTouch® operator to measure how steady the finger was held during the entire test period and to check how well the two stages of die tissue modulation process were executed. This training nearly always produced a test subject who was confident in his/her ability to execute an unpressed to pressed tissue modulation sequence. The entire training period never exceeded 5 minutes.

For this study, a measurement sequence consisted of 100 sec of unpressed and 100 seconds of pressed states. All testing occurred in the dark without benefit of any real-time feedback of any kind to either the LighTouch® operator or the test subject other than an audio cue to transition from the unpressed state to the pressed state. To test the difficulty in teaching/learning to execute a tissue modulation cycle, no test subject was ever given two chances to obtain a glucose reading. It is cleat, e.g. by use of pressure sensors, observing the deformation pattern on the fingertip skin and other measurements, that the applied pressure in the two states and the position of the measurement varies from one test subject to another. It is also possible to observe some kinds of unintended motion during the tissue modulation cycle by observing the blood volume versus time plot immediately after completion of the cycle. Using this plot the LighTouch® operator can select a set of unpressed frames and a set of pressed frames in equal numbers that are then co-added respectively before being subtracted, accumulated pressed from accumulated unpressed, to yield a tissue modulated fluorescence/Raman spectrum. In this way, at least some frames that were corrupted by unintentional motion or other sources of artifacts, e.g. obvious CCD spikes, could be excluded from subsequent processing.

The tissue modulation process as implemented in this study is not invulnerable to artifacts associated with surface imperfections on the size scale comparable to the physical extent of the laser spot and larger. Thus surface imperfections like cracking caused by excessive skin dryness, or trauma caused by physical injury, e.g. scatting due to long-term fingerstick blood glucose measurements, can be expected to lead to spurious raw data and therefore spurious blood analyte concentrations Although it is possible to use other fingers for measurements in some cases without recalibration, in this study only the middle finger was used. The data presented in the results section correspond to the results of every tissue modulation cycle regardless of the conditions of the test subjects' skin or peripheral circulatory or nervous systems. Patients were accepted for study based on their willingness to participate and their fingers were not examined before being invited to participate. The reproducibility of measurements on single individuals or simply people with similar skin condition makes us suspect that prescreening of subjects would lead to better results.

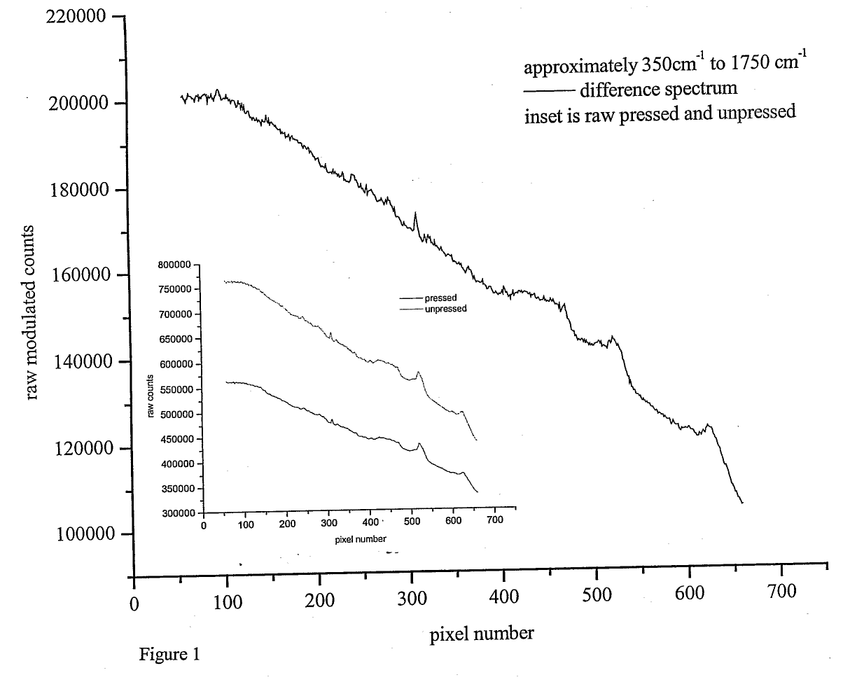

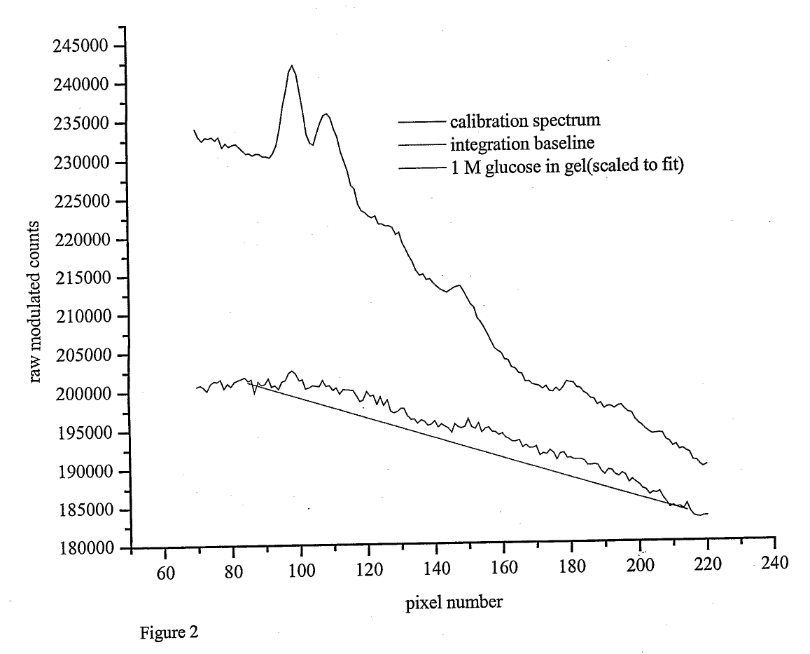

Extracting the glucose concentration from the tissue modulated spectra was accomplished in a similar but not identical manner as previously published. As can be seen in FIGS. 1 and 2 the modulated spectrum contains both fluorescence and Raman features. From those spectra and previously published results based on in vivo and in vitro spectra of authentic glucose, we have ascertained the wavenumber range containing the most glucose information balanced against the least tendency to contain off-axis Rayleigh scattered light. To illustrate the starting point for that procedure the spectrum of a large glucose concentration-spiked gel spectrum is included in FIG. 2. To obtain an integral over the same Raman feature(s), the same set of integration wavenumber limits found in out earlier studies were used as follows. First, a set of 10 pixels were averaged at each of the endpoints of the same spectral region to define the endpoints of a baseline. A straight line between these two points was used as the baseline. The taw tissue modulated spectrum was integrated down to the baseline between the endpoints as indicated in FIG. 2.

To obtain blood volume normalization for the spectra, each taw tissue modulated, i.e. difference, spectrum was integrated to zero from about 1000 cm⁻¹ of Raman shift to the highest shift accessible with the CCD detector which was usually about 1800 cm⁻¹.

Because the fluorescence constitutes the majority of the emission at all Stokes shifted wavelengths relative to the exciting wavelength, and because whatever amount Raman contributes to that emission is also a measure of the material that responds to tissue modulation, simply summing the raw counts in the difference spectrum, over the range least affected by unfiltered Rayleigh and other stray light, is sufficient to obtain a measure of the blood volume. This integral is then divided into the Raman feature integral shown in FIG. 2. We suggest that at least in principle, it should be possible to implement this essentially digital procedure in the analogue domain using optical filters and single channel detectors. In this case we might use one narrow filter for each of the 10-point endpoint averages, a third filter for the ≈350-≈600 cm⁻¹ glucose integration and a fourth filter for the ≈1000-≈800 cm⁻¹ blood volume measurement. A portable device designed to monitor only glucose might be possible using this scheme. Generally speaking, for multiple analytes, we would expect that dispersive optics, multi-channel detection and digital processing would be mote appropriate.

Previously we referred to the ratio of these integrals as glucose concentrations in “integrated normalized units”, or INUs. To calibrate the LighTouch® device INUs to mg/dl for human testing, a series of measurements, i.e. integrals of Raman features in blood volume normalized spectra, were paired with contemporaneous fingerstick glucose measurements in mg/dl. The resulting data pairs were plotted, fit to a linear regression and then inverted to yield blood glucose values in terms of subsequent ratios of integrals from identically processed data. We have found that such a regression based on data from one or more individuals yields a calibration that can be applied to anyone else.

### Results

The size of fingers varied by subject, as did the amount of pressure exerted by each subject, in either the pressed or unpressed stages. The position of the irradiated zone also varied because the tissue modulation aperture was oriented in the same manner for all the subjects but each subject fit in differently. Representative raw data from ≈350 cm⁻¹ to ≈1800 cm⁻¹ corresponding to the pressed and un-pressed states and their difference are shown in FIG. 1. FIG. 2 shows the ≈350 cm⁻¹ to ≈600 cm⁻¹ range as well as the integration range and the baseline that was used to obtain a glucose measurement. For comparison, ≈1800 mg/dl glucose in gelatin, arbitrarily scaled to allow easy visual comparison, is also shown. The representative taw data corresponded to a HemoCue® blood glucose of ≈100 mg/dl.

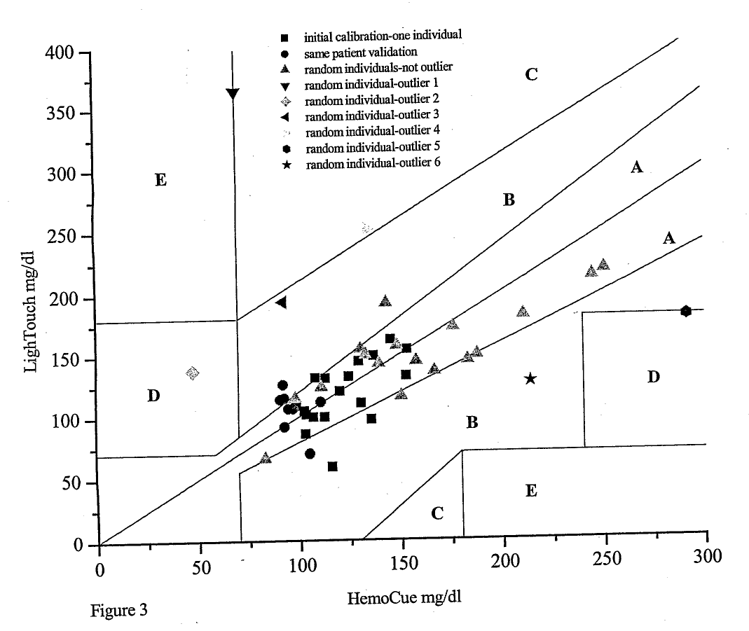

To calibrate the device, one individual (male, aged 68, 165 lbs. Caucasian, Type II diabetic) performed 18 fingersticks over two days to obtain the paired LighTouch®-HemoCue® data shown in FIG. 3. Although included in the data set plotted in FIG. 3, for calibration purposes one outlet was rejected based on a Q test (Shoemaker, “Experiments in Physical Chemistry”, page 41, 6^{th} Edition, WCB McGraw-Hill, Boston, 1996) and another on the basis of an instrumental inconsistency detected after the measurements were completed (wrong time pet frame was inadvertently selected). This was on a day when less than 10 points were collected and a small sample Q-test with 90% confidence limits was appropriate. The remaining points were fit with a linear regression that was subsequently inverted to yield a linear transform for the raw LighTouch® INU data to glucose in mg/dl. Over the next 14 weeks the same person contributed 7 additional measurements that were combined with single measurements on 24 different people. FIG. 3 contains the entire data set obtained from the 25 volunteers including the initial 18 measurements used to provide the concentration calibration in a Clarke Error Grid.

The error grid was developed (Clarke et al. Diabetes Care, 10, 622-628, 1987) to allow comparison of the performance of different kinds of fingerstick based devices since simple correlation coefficient and other statistical measures do not fully relate to clinical utility. Furthermore the zones allow for easier discussion of individual points. The zones in the grid are labeled with letters having the following meanings. Zone A denotes “clinically accurate”. Zone B denotes “clinically benign” or “acceptable” because such values lead to no treatment of the patient. Zone C is “unacceptable” because such values lead to over correction in the blood glucose level by the patient. Zone D denotes a “dangerous failure to detect and treat” and Zone E leads to “erroneous treatment”. The LighTouch® device produced 92% of measurements in the A and B zones. The remaining 8% of measurements were essentially wrong since they fell in the other zones.

Six of the erroneous measurements are color coded to permit tracking them through some but not all of the analyses that follow.

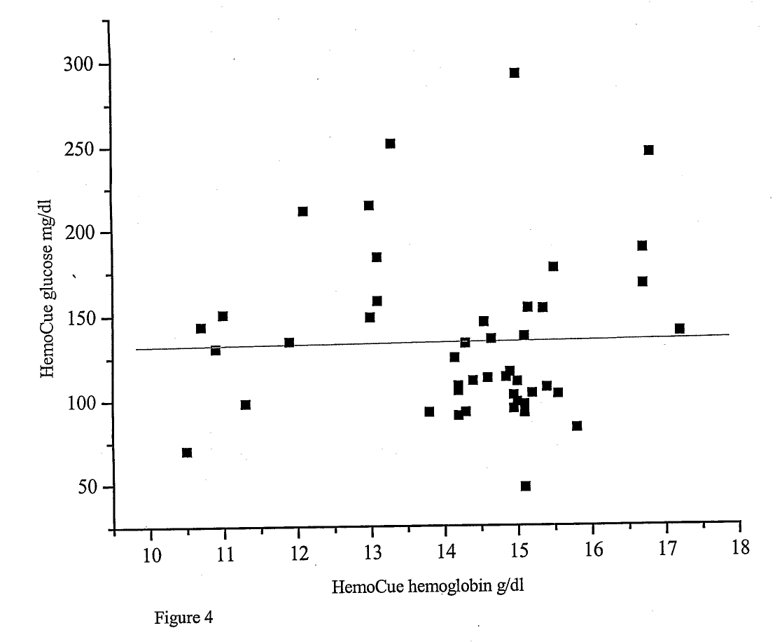

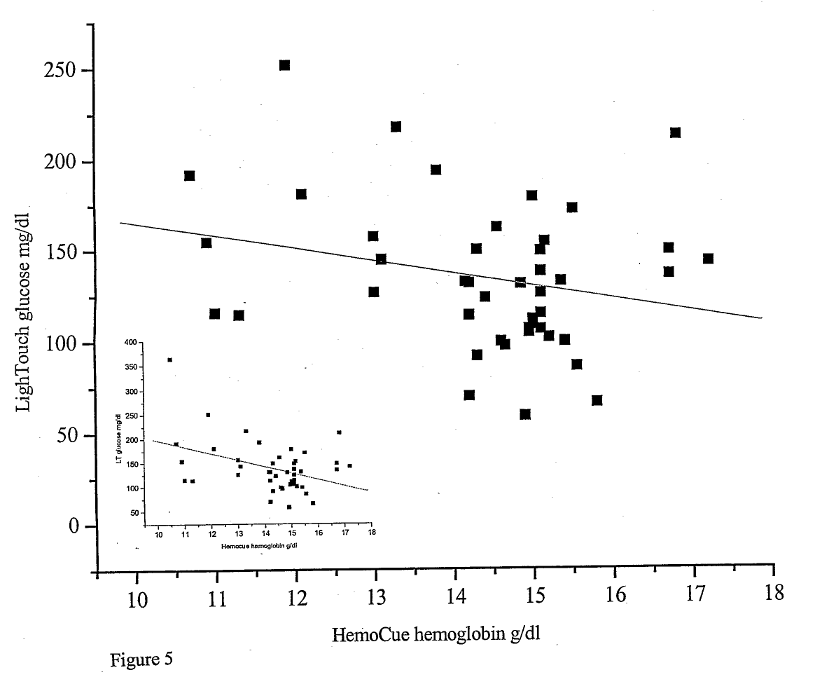

In all but 3 cases the test subjects consented to an additional fingerstick for the purpose of measuring their blood hemoglobin concentration. The average of the hemoglobin range observed in this study was 14.3 g/dl±1.6 (1σ) with a range of 10.5 to 17.2 g/dl. It is known that either reference device (i.e. hemoglobin or glucose) used in this study has systematic bias and less precision at the extremes of hemoglobin concentration. As can be seen in FIG. 4, there was no correlation between the HemoCue® glucose level with the HemoCue® hemoglobin level with r=0.016. We observe in FIG. 5 that the LighTouch® glucose level plotted against the HemoCue® hemoglobin measurement had a correlation coefficient of r=−0.42 when all points were included as in the inset. Note that there is a single point corresponding to LighTouch® glucose of 364 mg/dl that exerts a large effect on the linear fit. This point also corresponded to the lowest hemoglobin observed and corresponds to the single point in the E zone in FIG. 3. Without that point in FIG. 5 we obtain linear correlation of r=−0.27.

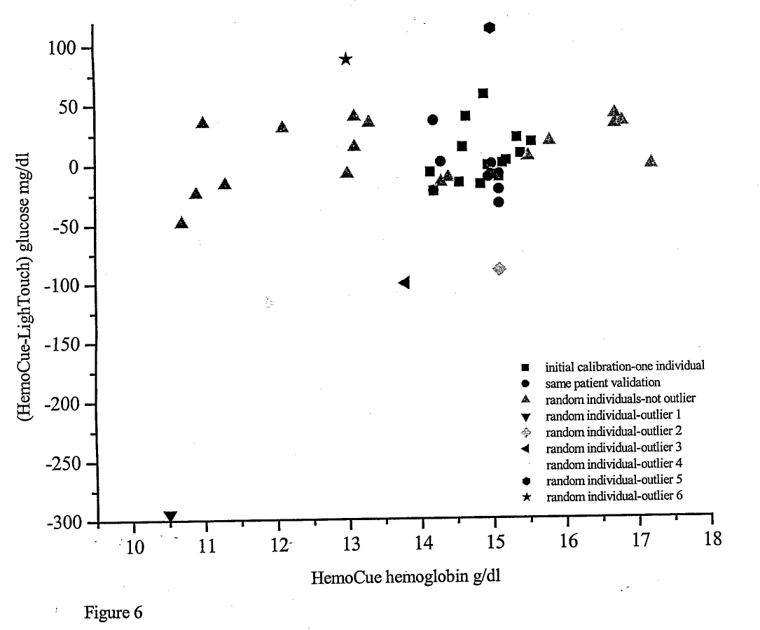

As must also be true based on FIGS. 4 and 5, the deviations between the LighTouch® glucose measurements and the HemoCue® glucose measurements in FIG. 6 are also weakly correlated with the hemoglobin concentration. The same E zone outlier point in all the above figures is evident and linear regression applied to whole data set produces r=0.38. Without that point there is a weaker linear correlation of 0.19 between the deviations and the blood hemoglobin concentration.

To further probe the role of the tissue modulation process itself in glucose concentration measurement, in FIG. 7 we plotted the deviations between LighTouch® and HemoCue® glucose measurements as a function of the “total modulated fluorescence”. The total modulated fluorescence is simply the integral of the tissue modulated light emitted from the capillary bed that is used for the blood volume normalization. Including all the data we find that the one E zone point again appears to be an outlier and including all the points linear regression produces a correlation coefficient of 0. Excluding this point yields a correlation of −0.19. The color coded erroneous points from the Clarke grid can be seen to form a ring around a central clump of points.

To probe the effect of varying the hemoglobin concentration on the tissue modulation process, we plotted the observed total modulated fluorescence as a function of the observed HemoCue® hemoglobin measurement in FIG. 8. Using all the data in linear regression, r=−0.07 and dropping the data points corresponding to the 4 highest modulations, which appear to be outliers, we obtain r=0.25. The data suggest that the maximum amount of modulated fluorescence increases with the hemoglobin concentration. We did not measure not attempt to control the amount of pressure applied by each of the test subjects and that would certainly be expected to have a large role in determining the amount of blood modulated. For a given hemoglobin concentration, pressing harder would be expected to move more blood and produce a larger fluorescence modulation. For all but one, which falls near the average hemoglobin concentration and the average modulated fluorescence, the color coded outliers from the Clarke Grid form a “boundary”, indicated by the line connecting those points near the top of the cluster of data points, regardless of the hemoglobin concentration.

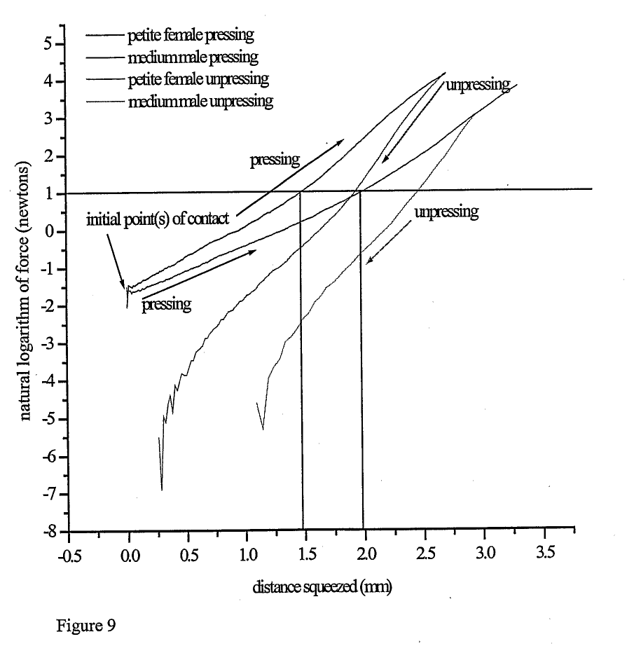

FIG. 9 shows the results for two different size fingers, each from a different person, of a measurement of total force as a function of displacement of the TA.XT probe from the position of first stratum corneum contact, as the probe is pressed towards the bone (distal phalanx). The pressure is seen to increase as the probe is brought towards the bone while the finger is being essentially squeezed between the probe and the testing surface. The probe is then backed-off rather quickly, 2 mm/sec. This produces a type of hysteresis because the finger does not re-expand as fast as the probe is backed-off from the position of highest compression. Based on the timescales involved, we assume that the blood and other fluids do not refill the capillary bed fast enough to maintain the total force experienced at the equivalent position during the squeezing cycle. Indeed, the flat surface of the probe and the stratum corneum adhere somewhat causing a negative force to be sensed by the TA.XT. The logarithm of the total force is plotted because it accentuates an inflection point that is always observed during the squeezing cycle. The position of the inflection point in both sets of data is marked by the horizontal line. The corresponding displacement for each of the inflection points is different because the gross size of the fingers is different. The petite young female displays the inflection point at a smaller displacement than does the larger male finger.

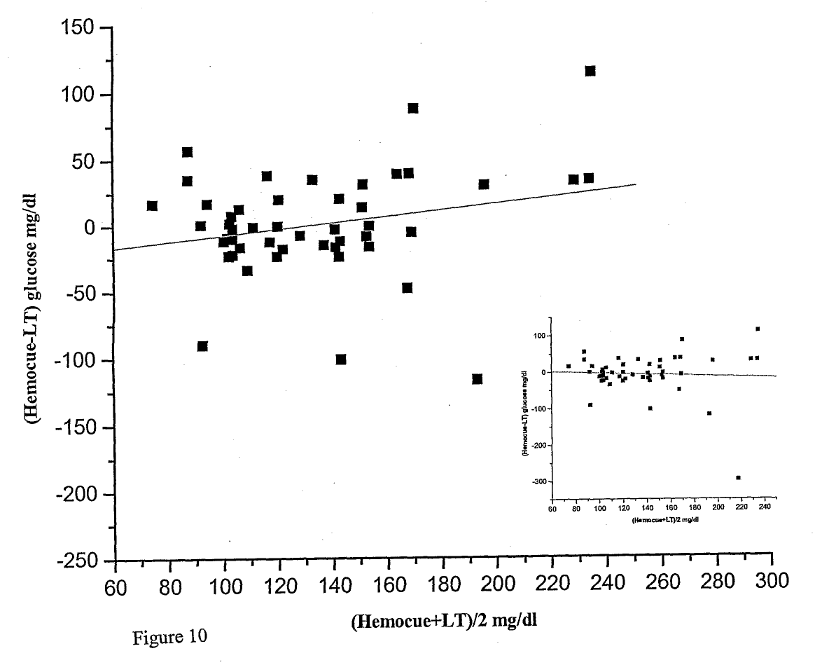

Since it is known that the HemoCue® itself has a bias at larger glucose values, we plotted the deviation between HemoCue® and LighTouch® as a function of the average between the HemoCue® and LighTouch® measurements FIG. 10. A significant correlation was not found.

### Discussion

The Clarke Error Grid analysis in FIG. 3 shows that 92% of the LighTouch® measurements occur within Zones A and B indicating that the LighTouch® device could be used to measure blood glucose by various individuals with a single calibration. Therefore, in general it is possible to obtain high quality glucose concentration measurements across the range of hemoglobin values and blood volume modulations sampled. To improve the performance of the LighTouch® device, the sources of the 8% of measurements that were erroneous need to be identified and if possible eliminated. We note that each concentration measurement is comprised of a blood volume measurement and a glucose measurement that to a large extent are independent of each other. The glucose based Raman signal is intrinsically much weaker than the fluorescence based blood volume measurement and to a large extent is much more spatially localized. Thus the two types of measurements are susceptible to only partially overlapping sources of errors. For just one example, the Raman signal is more susceptible to errors due to optical miss-alignment and other effects that can be traced to various sources. In what follows we shall attempt to account for the several outliers that can be seen in FIG. 3 on the basis of random and systematic errors.

As reported earlier (Chaiken et al., Proc. SPIE, Vol. 4254, 106-118, 2001), very small tissue modulations lead to low signal to noise. We suggest that very small modulations can be caused by either only a very small amount of blood actually being moved during the modulation process or because a reasonable amount of blood is moved but the hemoglobin concentration is much smaller than average. In the former case the Raman signal for glucose could have low signal to noise if only due to shot noise and so the concentration measurement will be compromised. The datum in the E zone likely corresponds to the latter situation since it also corresponds to an abnormally low hemoglobin concentration, the lowest observed in the study. As will be discussed below, in the latter case the Raman signal can have good signal to noise and the precision of the glucose concentration measurement would be expected to be good although biased in a systematic manner due to the variation in hemoglobin concentration. We do not think the E zone datum corresponds to some type of systematic error.

It is always possible to have an erroneous measurement at any particular hemoglobin concentration or blood volume modulation due to purely instrumental factors. No attempt was made to remove spikes from the CCD response during this study. True single pixel spikes can have only a very small effect on the blood volume measurements because hundreds of pixels are employed and the fluorescence is strong. Nevertheless, even a small spike in the region being used for glucose Raman measurement will usually be important. A later study should incorporate spike detection and removal on a per frame basis to minimize the effect of this source of error. Although we cannot be certain, a CCD spike in the spectral region used for the glucose Raman signal could easily be the source of the E zone datum.

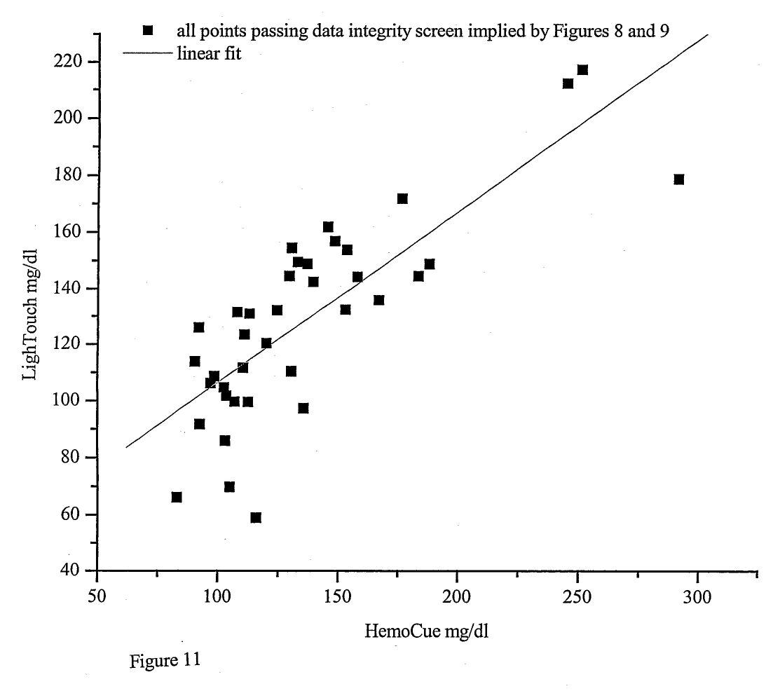

Taking into account the modulated blood volume and hemoglobin concentration in the manner described below, resulted in FIG. 11. In this case we reject only the five data points that form the “boundary” in FIG. 8 and the six data points having larger modulated blood volume. Rejecting these 11 points results in r=0.80, SD=22, for N=38. At least two of the rejected points are not outliers in the sense that since leaving them in actually improves the correlation and decreases the standard deviation relative to the two cases just described. As will be described below, we suggest that rejecting the 11 points on the basis of being above the boundary does not requite independent knowledge of the subject's hemoglobin concentration. It is our hypothesis that the 5 “boundary points” identified from the Clarke Error Grid analysis and the ones above the boundary points in FIG. 8 are associated with subjects who pressed too hard or too weakly during the pressed and unpressed tissue modulation stages respectively.

The deterministic consequences of pressing too hard or weakly are discussed a few paragraphs below however, if this is the case then measuring the applied pressure during the tissue modulation process and giving the “human servo” feedback in teal time should avoid this problem and improve the yield of good measurements pet attempts. Whether these suggestions are correct or not can be checked in a subsequent clinical study using a LighTouch® device that incorporates the above real time feature. Since the higher glucose values have a disproportionately favorable effect on the correlation, arbitrarily rejecting the three highest values results in r=−0.70 for N=35 and all remaining HemoCue® glucose values between 188 and 83. There were insufficient instances of HemoCue® measurements in the hypoglycemic range, i.e. <79 mg/dl, to assess the efficacy of the LighTouch® device in that range.

Torjman and co-workers (Diabetes Technolog and Therapeutics, 3, 591-600, 2001) report that for an inhomogeneous population in comparison with a laboratory plasma glucose measurement for reference (+0.1%), the HemoCue, in either the glucose range 79 mg/dl and below, as well as 79-140 mg/dl, has a correlation coefficient of 0.80. For 140 mg/dl and higher, r=0.97. Thus since about half of out measurements, 47%, corresponds to glucose values of 140 or below, and the LighTouch® must obviously have finite precision itself, in no case can we expect correlations much greater than about 0.80. Thus the observed values of the correlation coefficient for linear regression suggest accuracy and precision for the LighTouch® in line with current fingerstick technology. In this respect, we note the measurements of Glasmacher (Experimental and Clinical Endocrinology and Diabetes, 106, 360-364, 1998) among others in which the correlation coefficient for a number of FDA approved commercially available fingerstick devices ranges from 0.78 to 0.88 referenced to a laboratory method and all in the normal glucose range. It should also be mentioned that these devices have a small but measurable probability of producing C and D zone measurements.

The most important source of error in all fingerstick devices is undoubtedly human error. Human error undoubtedly played a role in this study since the tissue modulation process was not executed uniformly by all individuals. We have found from on-going studies that real time feedback to the test subject is very useful in alleviating this problem. One could also automate the process to attain greater uniformity and less error. Improvements that allow a decrease in the overall measurement time, such as increasing the laser power or the signal collection efficiency, would be expected to significantly decrease the rate of human error regardless of whether the tissue modulation is executed by the human servo or automatically.

The results of this study also suggest that there are subtle sources of small systematic error in the LighTouch® process. Although FIG. 4 reveals no correlation for this particular data set (r=0.02, N=46, p=0.92) between HemoCue® glucose measurements and hemoglobin measurements, linear regression on FIG. 5 suggests a weak correlation (r=−0.27, N=45, p=0.07 or greater) between LighTouch® glucose measurement and HemoCue® hemoglobin measurement. Essentially the same observation is contained in FIG. 6 suggesting a weak correlation (r=0.19, N=45, p=0.21) between the difference between paired HemoCue® and LighTouch® measurements and the hemoglobin concentration. The data suggests that, as expected, the LighTouch® tends to underestimate the true glucose concentration for subjects with a relatively high hemoglobin concentration.

To see why this is expected, we first point out that the LighTouch® uses modulated fluorescence emanating predominately from hemoglobin as a measurement of blood volume. To the extent that the skin does not change position or thickness during the modulation cycle, the skin contributes nearly no modulated fluorescence. If the hemoglobin concentration is above average then a given level of observed modulated fluorescence must emanate from a smaller than average actual modulated blood volume.

If the hemoglobin concentration is below average then the same given level of observed modulated fluorescence must emanate from a larger actual modulated blood volume. For a given actual glucose concentration, a smaller actual modulated blood volume will lead to a smaller glucose Raman signal thus leading to an under estimated glucose concentration. Combining this effect with the same reasoning applied to a smaller actual modulated blood volume signal accounts for the negative correlation observed in FIGS. 5 and 6. Note that the variation in hemoglobin concentration is about ±20% so the effect, while systematic, cannot be expected to be large.

FIG. 7 suggests that there is an optimal modulation, toughly between 1 and 3 E8 counts for the time duration, laser power, focusing conditions and other factors characterizing this particular LighTouch® device system. In attempting to understand and improve upon the tissue modulation process, we made no allowance for the fact that the fingers of all the test subjects do not have the same capillary density. The greater the density of capillaries in the irradiated zone (on average about 50 capillaries/mm²), the more blood that can be modulated. If all the test subjects had the same capillary density, and the capillaries had roughly the same size distribution with respect to the average erythrocyte diameter, then the modulated fluorescence should scale nearly linearly with the hemoglobin concentration. Of course, there is not necessarily any relationship between hemoglobin concentration and capillary density and this is reflected by in FIG. 8. Within limits, hemoglobin concentration can change on a daily or even hourly basis depending on various factors including a person's degree of hydration as affected by excess sweating due to physical exertion/stress or even eating salty foods or taking diuretics. Capillary density is an anatomical characteristic that cannot be expected to change much if at all on any daily or even weekly timescale. * Rough estimate based on capillary dimension data on page 1465 in Gray's Anatomy, 38^{th} Edition, Peter Williams, Editor, Churchill Livingston, New York, 1999 combined with loop-width data on page 545 in Geigy Tables, Volume 5, Editor C. Lentner, 8^{th} Edition CIBA-GEIGY, Basel (1990)

The lack of any relationship between hemoglobin concentration and capillary density is reflected by the fact that at any particular hemoglobin concentration there is a vertical range of possible modulation, “below the boundary”, that yields accurate glucose concentrations. We expect and observe that as the hemoglobin concentration goes to zero, so does the size of the modulation. According to FIG. 8, the lower (higher) the hemoglobin concentration the lower (higher) the maximum modulated blood volume that was observed to yield an accurate glucose measurement. Both the weakness of the observed positive correlation and the observed spread of the measurements even after rejecting possible outliers are consistent with the expected variation in physical size, capillary density and blood viscosity across test subjects.

In an earlier study we observed that such outliers were often associated with test subjects who had previously been diagnosed with conditions normally associated with peripheral vascular disease. By their own statements during testing, people with diabetic neuropathy cannot feel how hard they are pressing the aperture during either part of the tissue modulation cycle. The harder one presses, the greater the likelihood that the tissue modulation aperture will move during the measurement and, equivalently, the greater extrusion of skin into the aperture. Since the light collection system is designed accept light from die aperture in the plane of the tissue modulation plate, changing the position of the skin in either direction with respect to the plane of the aperture tends to decrease the amount of light collected. Therefore, a similar effect is expected during the unpressed stage if one does not press hard enough to maintain proper registration, i.e. the stratum corneum is behind the aperture. Either of these effects affects the collection efficiency of the Raman part of the concentration measurement in a manner different from the effect on die fluorescence measurement causing error and loss of precision in the glucose concentration measurement.

Taken together, FIGS. 8 and 9 suggest that by measuring the applied pressure during a tissue modulation cycle, it will be possible to automatically compensate for differences in the size and capillary density of different test subjects. The slight inflection in the measured pressured with increasing pressure defines a point where the blood has been removed from the tissue modulated region and simple compression of the remaining static tissue is the only process that remains. By maintaining the applied pressure below this level, that is approximately equal for different test subjects, and corresponds to a different displacement of the tissue modulation aperture with respect to the surface of the stratum corneum, extrusion of tissue through the tissue modulation aperture and gross movement of the aperture itself can be minimized if not avoided completely. This approach is being implemented and will be tested to see if out hypotheses are correct in the next generation LighTouch® device.

Torjman (Diabetes Technology and Therapeutics, 3, 591-600, 2001) had previously observed that when HemoCue® glucose measurements were compared with reference glucose measurements using plasma, there is a slight bias towards HemoCue® underestimating the glucose concentration at high glucose concentrations. This observation motivated the comparison associated with FIG. 10 analogous to the comparison made by Torjman and coworkers. Regardless of whether the average of the HemoCue® and LighTouch® measurements or just the HemoCue® measurements are used in the abscissa, an analogous small positive correlation with respect to linear regression is observed.

The cumulative data reported show that the LighTouch® measurement system can be used to monitor blood glucose in the normoglycemic range with accuracy and precision commensurate with current FDA approved fingerstick glucose meters. Using totally noninvasive measurements we have reproduced weak dependences that have already been established for blood using conventional invasive techniques. This could only be the case if the tissue modulation process was indeed providing quantitative spectroscopic access to blood and blood glucose. Furthermore, a single calibration based on one individual is applicable to random people and is also stable for a period of months. Continued effort towards improving and evaluating the LighTouch® device is clearly justified. Quantitative vibrational spectroscopy of human blood is feasible as a clinical research tool for experimental and practical metabolic monitoring involving various analytes and may also become useful for the self-monitoring of blood glucose by people with diabetes. To support this conclusion we cite the excellent work of Puppels and coworkers (Caspers et al., Biophysical J., 85, 572-580, 2003) who have recently obtained noninvasive blood spectra in vivo and noted the potential for glucose sensing using confocal Raman microscopy.

### CONCLUSIONS

We have established a method for quantitative noninvasive vibrational spectroscopy of human blood in vivo. The method produces glucose concentration measurements with accuracy and precision comparable with FDA approved fingerstick devices and therefore can be expected to contain information bearing on various other analytes. A single calibration of the device using data from one individual is applicable to other randomly chosen individuals and is stable for a period of at least ≈3 months. We have established sources of random and systematic error in the process. At least one source of systematic error is associated with hemoglobin concentration variation while random error may result from inadequate registration of the fingertip with the tissue modulation aperture at either stage of the modulation process. The results suggest that measuring the applied pressure and providing real-time feedback to the test subject during the tissue modulation process will improve the success-rate of the measurement process even without prior knowledge of a test subject's hemoglobin concentration. Although there may be other sources of error and loss of precision, both systematic and random, satisfactory management of the tissue modulation process could result in a device for the clinical and self-monitoring of blood glucose and other analytes.

### EXAMPLE 2

### Hardware and Software Implementation of an Optical Grip™

This Example illustrates implementation of an ergonomically feasible tissue modulator that is easy to execute. To this end, we have designed an “Optical Grip™”. One embodiment is pictured in FIG. 12. One can see that this grip fits the hand nicely and transforms the pressing-unpressing sequence into a grip-ungrip motion. We have found this to be much more comfortable and easier to maintain for the measurement period. Although we strive to make the measurement period as short as possible, and fully expect the eventual period to be under 30 seconds, the easier it is to execute, the easier it will be for people to hold steady during the measurement process. A few other embodiments are shown in FIG. 13.

The embodiments shown in FIGS. 12 and 13 are aluminum castings, but the preferred embodiments comprise cold-formed epoxy or other plastic material. The embodiments shown in FIG. 13 illustrate a layout for optics and electronics. An automatic Optical Grip™ in which the tissue modulation process is implemented without involvement of the patient, e.g. for unconscious patients, can be envisioned from viewing the embodiments shown in FIG. 13. It should be pointed out that the grips pictured actually fit a broad range of hand sizes, but it is not necessary to produce many sizes to encompass the vast majority of humans.

While these pictures (FIGS. 12-13) show how to make the tissue modulation process more ergonomic, the invention further provides a means to include the real-time feedback process into the Optical Grip™ paradigm. First, a multitude of commercial off the shelf (COTS) “pressure sensors” are easy to find and those skilled in the art can select a device in accordance with desired pricing, quantities, performance and size characteristics.

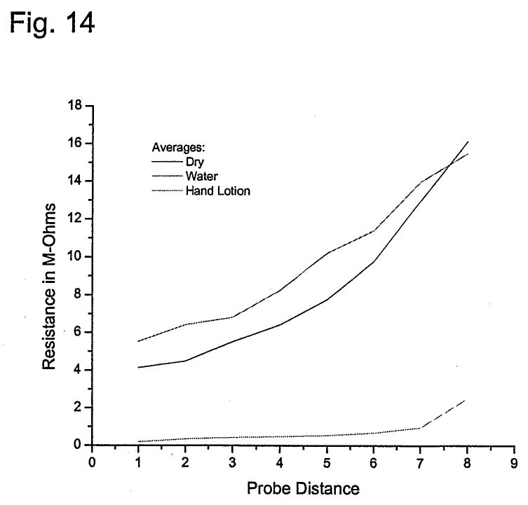

Position sensing is also easy to envision by various approaches. One is directly suggested by the graph shown in FIG. 14. The graph shows the measured resistance between two electrodes connected by a finger placed over the electrodes. The finger was either dry, i.e. without added water, moistened and then dried with a paper towel or mildly lotioned with all excess being toweled off. In this case, we envision a set of metal pads which are contacted in a specific manner when the skin is placed in the proper position with respect to the tissue modulation aperture. In that case, electrical circuits are closed, resistances go from infinite to finite, and the computer senses these changes in real time. If the patient moves during the process from the proper position, real-time feedback is provided to correct the position. When the measurement cycle is completed, the CCD frames corresponding to bad positioning can be discarded from the glucose concentration calculation.

Using this approach, we also get a measurement of the position. We also can derive a measurement of the degree of hydration of the skin. A measurement cycle can be declined by the LighTouch® device before anything happens if the skin resistance is not within a specific predetermined range. Note that there are time and geometrical dependant aspects to this measurement, as we are actually measuring an impedance. In this way, we simultaneously determine proper positioning of the capillary bed with respect to the tissue modulation aperture and also a measure of the degree of hydration of the skin to be traversed.

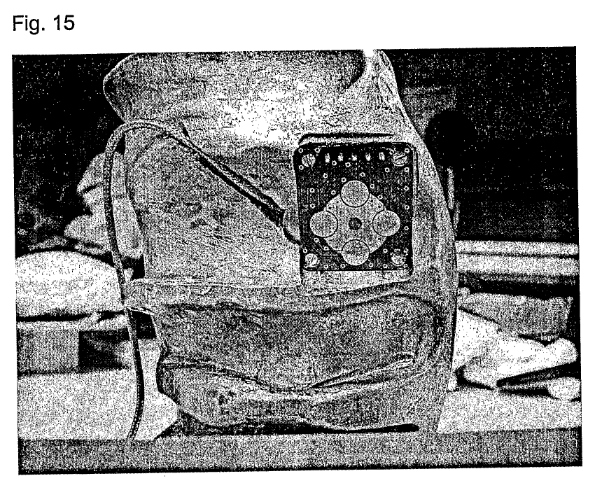

We have built a digital signal processing board as well as expanded our software system to incorporate all of the measurements into the tissue modulation process. An Optical Grip™ that incorporates all the aspects of the complete system is shown in FIG. 15.

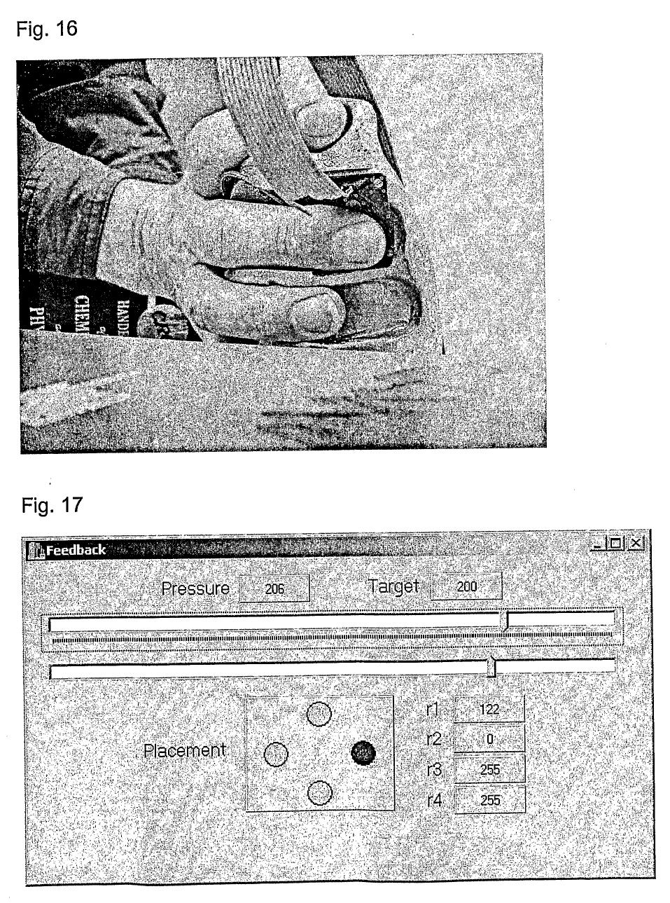

The printed circuit board (FIG. 15) incorporates four electrodes surrounding a central hole that serves as the tissue modulation aperture. A pressure sensor can be seen with its coax coming off to the left. A picture with a hand/finger in place can be seen in FIG. 16.

Although there are a variety of ways to format the real-time feedback, currently the reporting software presents the window shown in FIG. 17. There is a pattern that resembles the electrode pattern where each spot is green if it is properly contacted and red if it is not. Furthermore, there are numbers next to each spot giving a numerical value to the actual moisture content. The user can decide if these values are in range. In the long run, this can be completely automated. In the case shown, one electrode is not contacted properly and the patient would need to shift his/her finger to make all four spots green.

The pressure is indicated by the sliders across the top of the Feedback Window. The target for the pressed stage in this case is 200 units. These units can be calibrated in absolute Newtons using a procedure we have already devised. As the patient presses the tissue modulator aperture, the top slider moves in real time while the target slider stays constant. The patient then tries to make the two slider positions come into registration as shown. A numerical readout is also provided. When the tissue modulation cycle changes from pressed to unpressed, for example, the target will change and the patient will track.

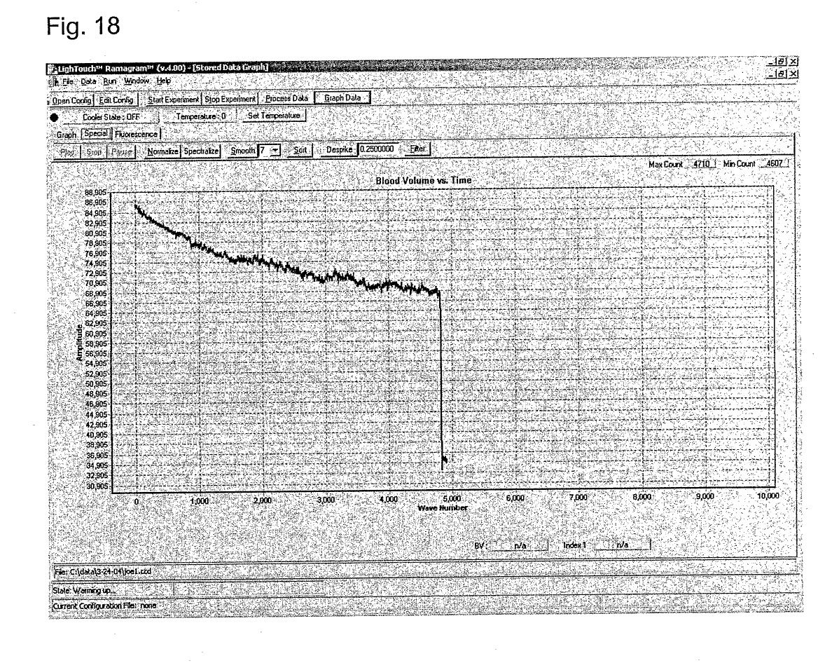

The hardware and software will track the readouts of the position and pressure sensors as well as the CCD data throughout the tissue modulation cycle. At the end of a cycle, all the data will be stored in the computer and can then be sorted. These data can be viewed in a time correlated, manner so that there will be three windows tiled on the computer screen corresponding to position, pressure and CCD response. The CCD response appears as the blood volume versus time window shown in FIG. 18 (wave number in this figure actually refers to frame number). In this case, we show the value of the normalizing fluorescence integral as a function of CCD frame number. The tissue modulation pressure and position will be tiled below. The frames can be sorted on the basis of any of the criteria, i.e. pressure, blood volume or position. Ultimately, this function will be completely automated. For clarity, only the blood volume versus time is shown in FIG. 18. The sorting is color-coded and mouse controlled. Once the frames are chosen, all the red frames are co-added from which the co-added yellow frames are subtracted. This yields a tissue modulated spectrum that can be processed for a glucose concentration.

### EXAMPLE 3

### Data Collection Using an Optical Grip™

This Example illustrates information obtained from a subject using a grip of the invention, including the effects of unexpected movement by the subject and how spectra associated with blood glucose can be obtained.

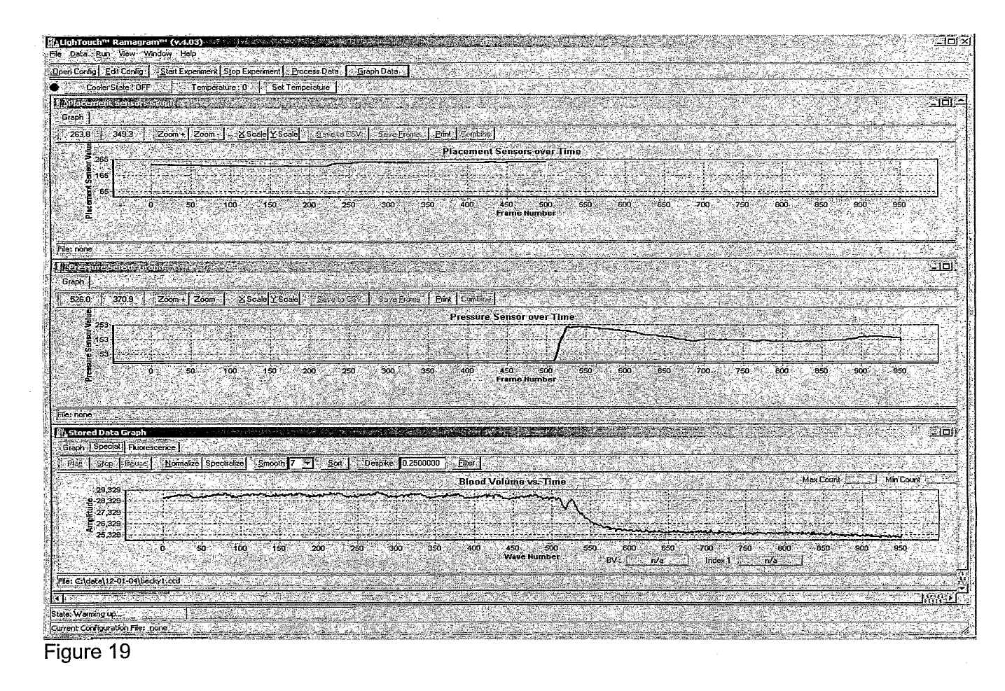

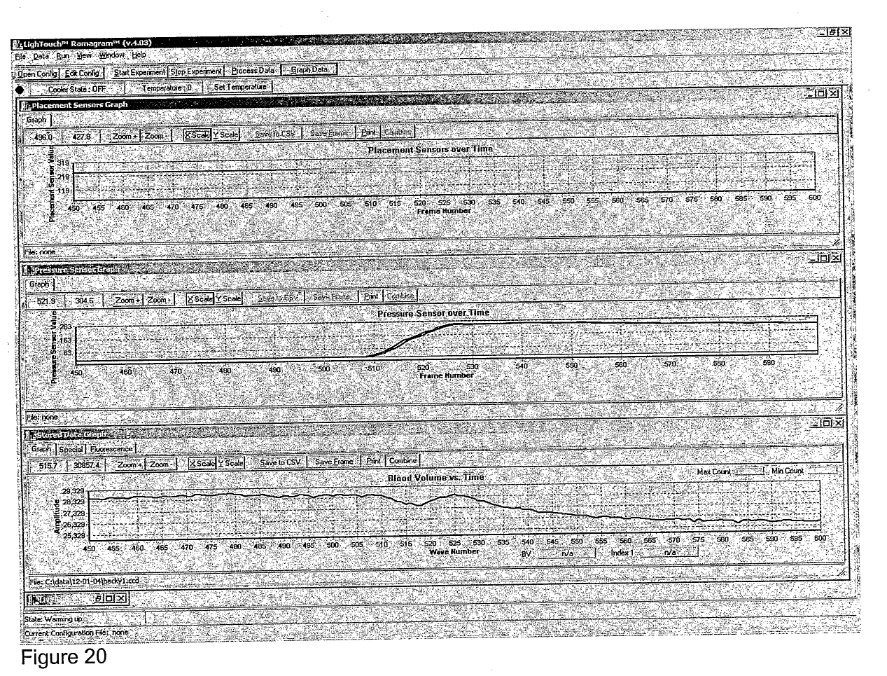

FIG. 19 shows a window (from a computer screen) containing three panes representing a staged 20 second test. The top pane shows the color coded response of the position sensors. The greater the value on the ordinate, the more physical contact there is between the associated sensor and the fingertip. Dynamic range and magnitude of response can be further calibrated to allow simultaneous moisture estimation. There are four sensors, and only three responses can be seen in the pane, so one can conclude that the finger is not stationary or in perfect position for the measurement. Note that each sensor is sampled twice at about each 20 msec, i.e. each frame is 20 msec. The pressure sensor is on the middle pane and the pressure change (about 150 grams) during the modulation is clearly visible starting at about frame 510 and concluding at about frame 525. After that the test subject attempts to maintain a relatively high pressure, but there is a slight variation that is also reflected in the blood volume measurement. The blood volume vs. time plot is the bottom most pane (please note that the abscissa is mislabeled as “wavenumbers”—it should be “frame number”), i.e. the integrated fluorescence as a function of time, and it also clearly shows the intentional modulation at frame 510. The pulse can be clearly seen during the unpressed stage as well.

Perhaps one of the most telling observations in the staged 20 sec test, is the “flinch” at frame 517. The pressure sensor data is also sampled twice pet 20 msec frame. The pressure data is presented as two measurements pet frame (the position sensor shows the average of each pair of measurements). FIG. 20 shows the same three panes between frames 450 and 600 to more clearly show the tissue modulation transition. In this expanded figure, the flinch in the pressure at frame 517 is clearly reflected in an inflection “bump” in modulated blood volume starting at frame 517 but extending a little further in time. Note that, when a color change is visible in the pressure dependant data, the software is noting that the measurement is out of a preset range. In this case, such a section was simulated at about frame 520. After full calibration, this feature can be used as a precursor to an automatic data sort for producing tissue modulated spectra. Thus it is possible to use this combination of sensors to distinguish between position, pressure and blood volume variation.

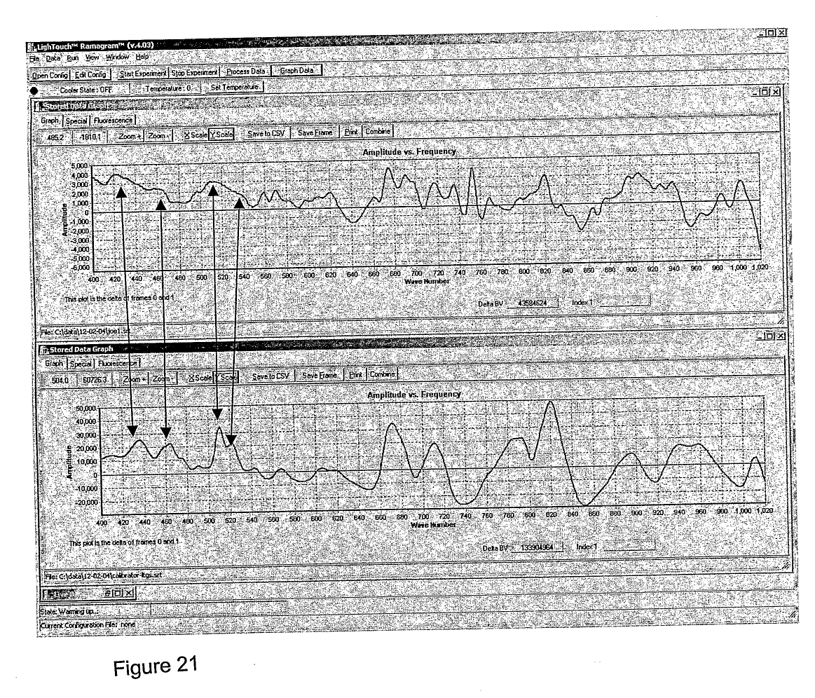

Finally, the resulting tissue modulated spectrum (note uncalibrated wavenumber scale) obtained from a full 200 second tissue modulation cycle can be seen in FIG. 21. The raw tissue modulated spectrum, accumulated unpressed frames minus accumulated pressed frames, was subjected to the same baseline removal procedure (as in our previous publications) giving the spectrum of blood shown in the FIG. 21. Arrows are included to show corresponding spectral features in a glucose containing calibrator spectrum (lower spectrum) that are used to analyze blood for glucose concentration.

### EXAMPLE 4

### Ergonomic Considerations for an Optical Grip™

The role of the grip is to transform the application of force needed for tissue modulation from a flex of muscles distributed throughout the entire hand, forearm and upper arm into a flex of muscles located in and around the hand and fingers. Making this transformation is ergonomic and allows use of muscles more capable and therefore appropriate for the task(s) of fine coordination and placement.

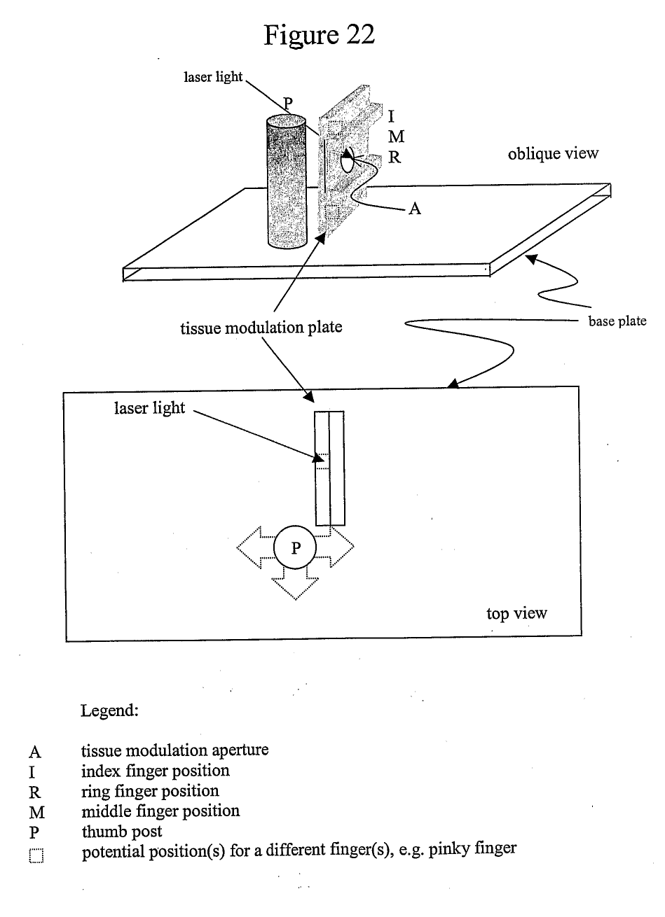

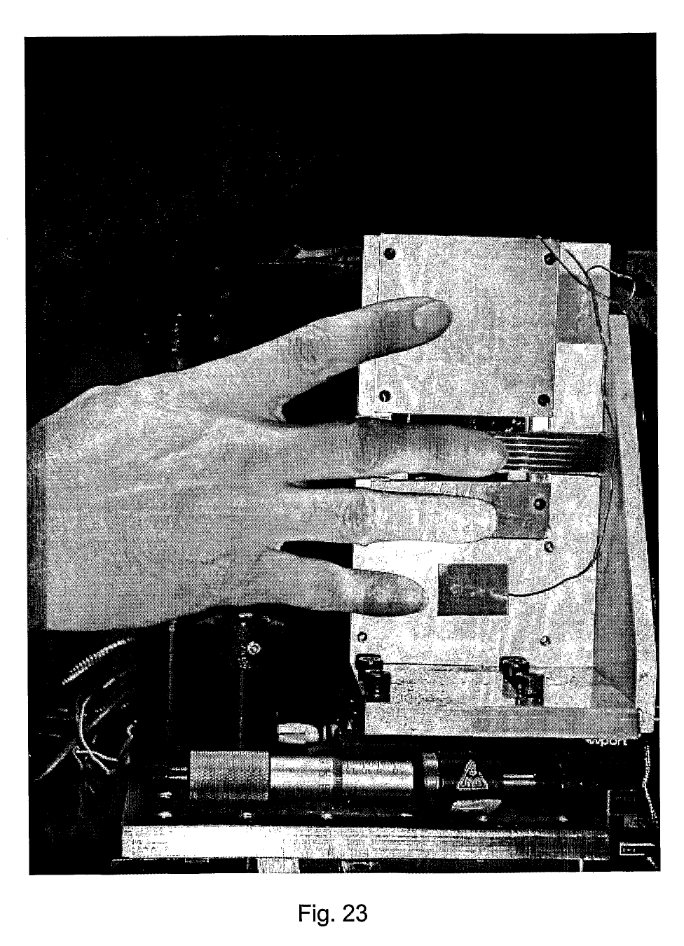

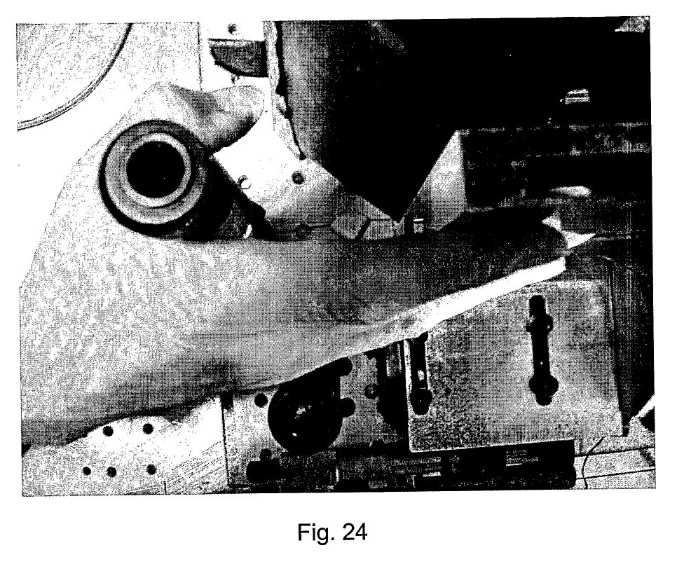

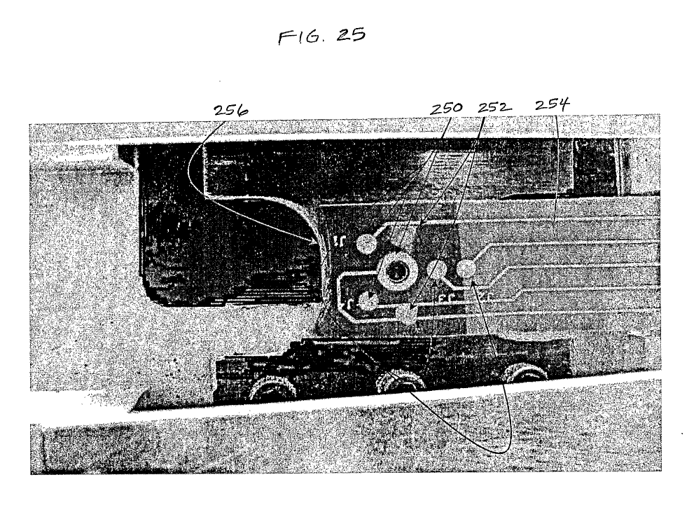

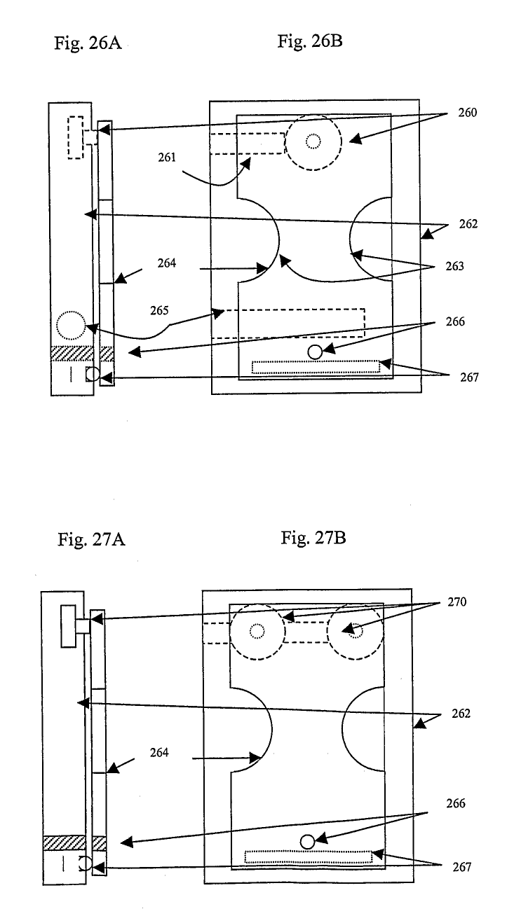

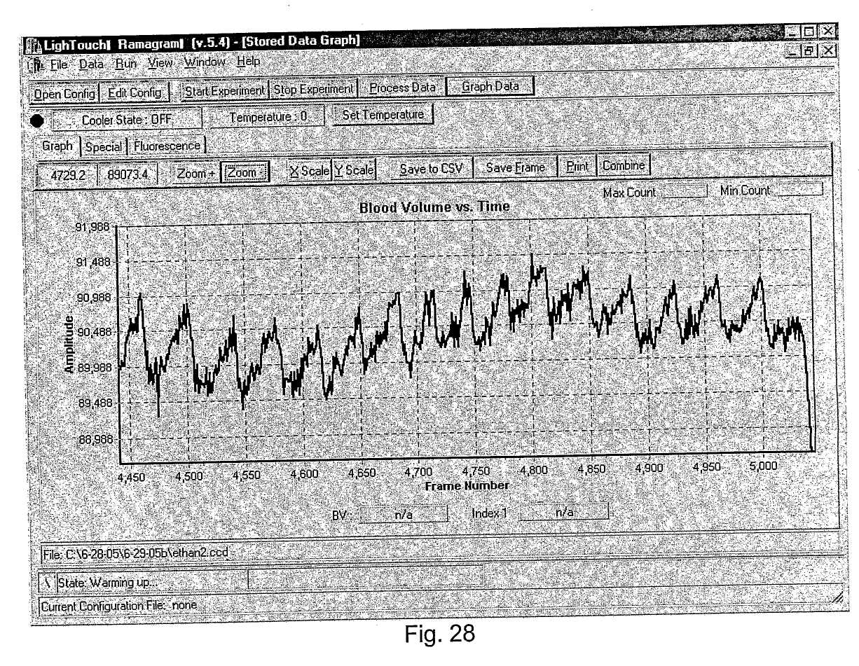

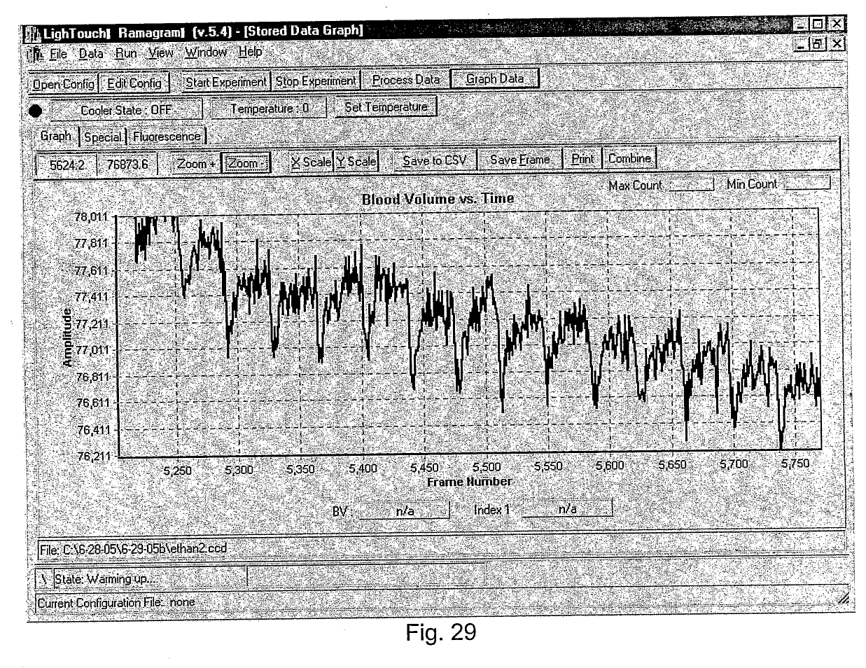

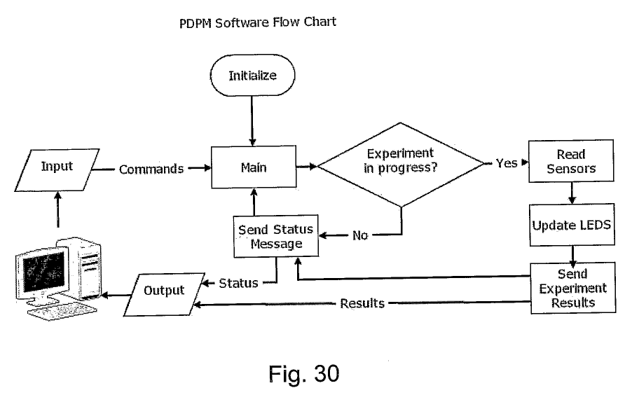

FIG. 22 illustrates an idealized grip, indicating the relative positions of the subject's fingers and the aperture through which laser light passes. The positions indicated for the various fingers are suggestive of their differing roles in the overall process, thereby making it easier for the test subject to comply with the tasks being directed by the real-time feedback unit. This unit utilizes pressure, position, moisture and possibly other sensors embedded in the tissue modulation plate, as indicated by a thin black line on the edge of the tissue modulation plate in the oblique view. There can be many possible shapes of grips that can accomplish the desired transformation, but they are variations on the idealized grip shown in FIG. 22. Included is a surface, the “thumb post” (P) in FIG. 22, that will allow the user to anchor their thumb into a specific position. Because hands and fingers are different sizes and shapes, it will often be helpful but not absolutely necessary, that the position of the post be adjustable, perhaps as indicated in the top view of FIG. 22. Also included is a tissue modulation plate that will allow the test subject to bring their fingertip into proper registration with the tissue modulation aperture, thereby allowing it to be probed by the laser light.

### EXAMPLE 5

### Optimization of Noninvasive, In Vivo Collection and Analysis of Raman Spectra Obtained from Human Blood Plasma

Because of this invention we have become much more precise in ensuring that there is uniform and consistent finger placement in the tissue modulator as well as acceptable moisture content in the skin. Directly because of these improvements, we have discovered and characterized an important effect in tissue and pulse modulated Raman spectroscopy of human tissues in vivo. The ramifications of the well known plasma skimming, Faraeus and Faraeus-Linqvist effects in the context of tissue modulation allow spectra of blood plasma to be obtained in vivo, non-invasively. “Skim pool” spectroscopy can be performed with good precision and reproducibility by carefully measuring the tissue placement and force applied during tissue modulation in conjunction with using that information to supply teal time feedback to either the test subject or to appropriate electromechanical and other devices.

In addition to the issue of human factors, the simultaneously measured hemoglobin concentrations allowed a comparison of the residuals between the spectrometrically estimated glucose concentrations and associated finger stick estimates of glucose concentration. We had previously shown that a major source of fluorescence in blood is from hemoglobin and so we had been using the modulated fluorescence as a measure of modulated (tissue) blood volume. Thus it is possible that variation in hematocrit, red blood cell count (RBC) or hemoglobin content per RBC could cause variation in our estimate of modulated blood volume. Systematic contributions to imprecision and inaccuracy in measured analyte, e.g. glucose, concentrations resulting from person to person variation in hemoglobin concentration, or from time to time for an individual are therefore possible. Because of our improved precision in the tissue modulation process we will shed some light on how some systematic effects occur in this paper.

Finally, use of pulse modulation allows spectra to be obtained under the least invasive or otherwise disruptive circumstances. Potential effects on spectra due to pressure induced index of refraction, reflectance, layer thickness variation, or even small motion of the tissue in the effective light irradiation/collection volume are minimized if not eliminated. In earlier work3 we showed the effect of plasma skimming and the Faraeus and Faraeus-Linqvist effects on the spectra by noting the decreased hemoglobin Raman features relative to NIR Raman spectra obtained in vitro using blood that was sampled from a relatively large vessel. However, it will be shown that even this process must be executed with accuracy and precision or clinically useful information will not be accessible. We show that it is possible to closely integrate the tissue process with the processes that are known to occur in the microcirculation to effectively control the effect on hemoglobin concentration variation on the blood volume estimate.

### Experimental

Less than 45 mW of light at either 785, 805 or 830 nm from a Sachet Laser (Sachet Laser, Marburg, Germany) is used to interrogate the volar side of the middle finger of the tight hand of the researchers. A Semrock (Rochester, N.Y.) filter is used to “clean up” the laser light before it impinges on the tissue through a small hole in a piece of thin spring steel at an angle of about 51°. We have previously shown that fused silica must be used in the delivery optical train so as to minimize optical system fluorescence. The spot size is not circular, but about 100 μm in diameter. This light is collected and collimated using a commercial off-the-shelf Melles Griot fused silica lens with NA=1.4. The collimated light has much of the Rayleigh scattered light removed while traversing a Semrock Razor Edge filter before being focussed onto a bundle of fused silica fibers. The fibers present a round target for the filtered collected light but are configured to introduce a line image into a Process Instruments (Salt Lake City, Utah) f=1.4 spectrograph downstream. This spectrograph presents a flat focal plane to a −80° C. cooled 256×1024 Andor (Andor Technologies, South Windsor, Conn.) CCD camera. Typically, we employ 20 msec CCD frames in full vertical binning.

The tissue modulator used in the study contains many improvements over earlier prototypes. To appreciate these improvements it is necessary to briefly review the basic idea of the tissue modulation process. For first stage, i.e. 100 sec, of the process, the test subject tries to place the volar side of the distal segment of a fingertip in registration with the aforementioned hole in the spring steel while avoiding either extraneous motion or applying too much force between the fingertip and the spring steel. For the second 100 seconds the test subject is directed to press against the hole in the steel applying a constant amount of force directly perpendicular to the steel surface. The point of this sequence is for the LighTouch® device to accumulate spectra during the un-pressed stage and then to accumulate more spectra during the pressed stage. The sum of the accumulated pressed spectra is subtracted from the sum of the accumulated un-pressed spectra to obtain a spectrum of whatever material moved or was “spatially modulated” by the applied force. By many types of experiments we have shown that the spectra obtained in this way are comprised predominantly of the most mobile fluid in the irradiated tissue, i.e. the blood. Furthermore, the integral of the total emission is a measure of the blood volume present in the overlap volume of the irradiation and the collection optics.

The latest LighTouch® device responds to the observations inherent in the results of the small clinical study by providing teal time feedback to the test subject during all phases of the tissue modulation process. As in out last LighTouch® prototype, there are rubber Braille-like “nubs” arranged near the tissue modulation orifice/aperture that allow the test subject to orient the position of his/her fingertip to the hole by way of touch. Now, the spring steel is also a substrate for a specially design flexible, i.e. Kapton®, circuit board that presents conductive, i.e. metal, surfaces that must be contacted when the test subjects' skin in placed in the proper position with respect to the aperture. The LighTouch® detects the electrical resistance between the surfaces which is minimized, i.e. falls below a preset threshold, when the skin surface provides a connection between them all as when the finger is properly placed relative to the aperture. The electrode pattern is mimicked by a set of light emitting diodes (LEDs) that vary between green or red in real time depending on the position of the test subject during the tissue modulation, i.e. un-pressed-pressed, process. Thus the LighTouch provides teal time feedback to the test subject allowing for consistent and steady finger placement during each tissue modulation cycle and from time to time.

The device also employs a special load cell to measure the force applied to the spring steel by the fingertip during the different stages of the tissue modulation process. Trial and error has shown that the aperture must move very little if at all during the tissue modulation cycle if consistent results are to be obtained. The amount of force needed to affect an “un-pressed”, i.e. the “target” force, is about 3-6 grams while the amount or “target” needed to affect a “pressed” is greater as will be discussed more thoroughly later. The LighTouch employs a linear array of several LEDs in a pattern of red, yellow, green, yellow, red. During the tissue modulation cycle, the LighTouch gives an audio cue to the test subject while causing the LEDs to light-up depending on the test subject's behavior. During the un-pressed stage, the test subject must apply about 3-6 grams in order to cause the middle green LEDs to be lit. Too much or too little and the red and yellow LEDs on either side of a center green pair respectively inform the subject of the discrepancy. In this way the LighTouch® provides teal time feedback to the test subject.

Finally, the position sensors use the skin conductivity in a threshold mode but the LighTouch® monitors their absolute values to estimate the moisture content of the skin surface. Experience has shown that when the skin surface is too dry and cracked the scattering of light is detrimental to the measurement process. Thus at the outset of each measurement cycle the LighTouch® compares the measured skin conductivity with preset benchmarks to inform the test subject of the possibility of an impending bad measurement cycle. Based on this information the LighTouch® operator can choose to abort the cycle or perhaps to direct the teat subject to use a skin moisturizer of some kind.

The new LighTouch® prototype therefore is much more precise in ensuring that there is uniform and consistent finger placement in the tissue modulator as well as acceptable moisture content in the skin. Moreover, the force applied in the pressed and un-pressed stages can be measured, documented, analyzed and optimized. The LighTouch® stores this information, sampled at 50 Hz, for every measurement cycle. A LighTouch® operator or the instrument itself can be programmed to accept or reject a measurement based on the information contained in these variables. The information base being compiled using this prototype is the basis for implementing a completely automatic tissue modulation device in which the measurement cycle is implemented and managed by an electromechanical system that is integrated with the spectroscopic system.

To obtain consistent measurements the applied force should be monitored to within about ±2 grams. The aperture must be stationary to within at most about ±25 μm with respect to the fixed positioning of the optical system. The capability to remain completely motionless or to apply constant force varies widely from individual to individual and in some cases for the same individual at different times. Nevertheless 100 sec of acceptable “un-pressed” and 100 sec of acceptable “pressed” condition can be maintained by most people most of the time. For most test subjects, due to the heart pulse, it is usually possible to observe pulses of emission under constant irradiation conditions. If there is too much motion then the pulses become difficult to observe.

Some fluorescence spectra were obtained using a Photon Instruments spectrometer on human blood in vitro. The blood was donated by one of the authors under the supervision of a local physician. Plasma was obtained by centrifuging blood collected directly into heparinized tubes. Serum was obtained by gently centrifuging blood collected into serum tubes. Samples of the hematocrit were added to either deionized water or deuterated phosphate buffered saline and then spun down before spectra were obtained. As described previously some experiments¹¹ were conducted using a TA.XT2 texture analyzer (Stable Micro Systems, Surrey, England) in which the applied force could be measured simultaneously with a time stamp and the displacement of the aperture.

### Results

The first experiments with the new tissue modulator entailed exploring the choice of parameters, e.g. the applied force in the two different tissue modulation stages, to more closely define the overall tissue modulation process. Initially the real time feedback was turned off, the authors were the test subjects, and we attempted to perform the tissue modulation process in the same manner as we had in our earlier study. Without the feedback we simply measured the force that people applied. It was found that all varied widely in what was considered pressed and unpressed. Given this new objective characterization of what was previously a subjective judgement we decided to systematically vary the force requited by the teal time feedback system for the two different conditions.

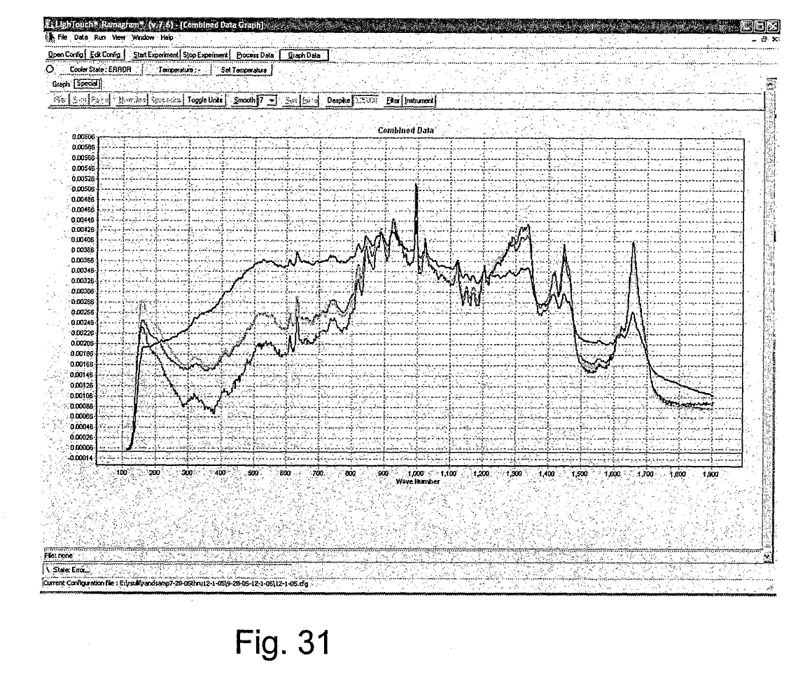

The un-pressed condition was chosen to be the smallest force that most people can apply consistently. The blood is so mobile that if the un-pressed condition is chosen too high, there is little blood remaining to modulate when the pressed condition is requited. The pressed condition was explored by choosing a constant un-pressed condition and varying the force demanded by the real time feedback system as the pressed condition as shown in FIG. 31. The spectra shown are all from the same individual using the same fingertip—with physical activity and between measurements to allow the subjects circulation to equilibrate between trials. As the “pressed” condition target force is increased, the tissue modulated difference spectra are seen to lose counts below several hundred wavenumbers of stokes Raman shift relative to larger Raman shifts. This effect was observable in the spectra of all volunteers although the values of the force needed to observe a set amount of depletion in the low wavenumber region varied from individual to individual. For all volunteers, if the applied force is large enough, then the depletion does not increase further and in some cases was seen to decrease. When the spectra are normalized to the integrated total counts above 800 cm⁻¹ we obtain FIG. 31. Many Raman features are seen to become larger relative to the apparent fluorescence although there are spectral regions where all the spectra have nearly the same normalized emission. For example, the vicinity of ≈1560 cm⁻¹ of Raman shift is the most characteristic region of porphyrins without having overlap with features associated with other entities.

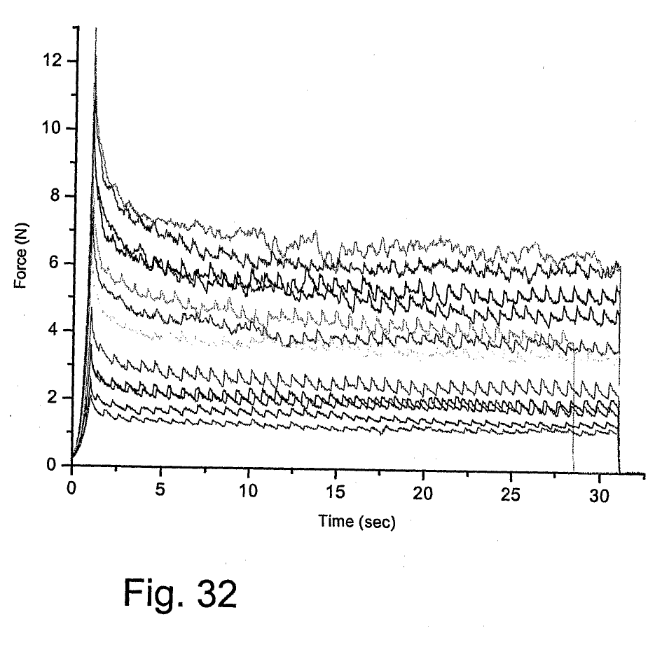

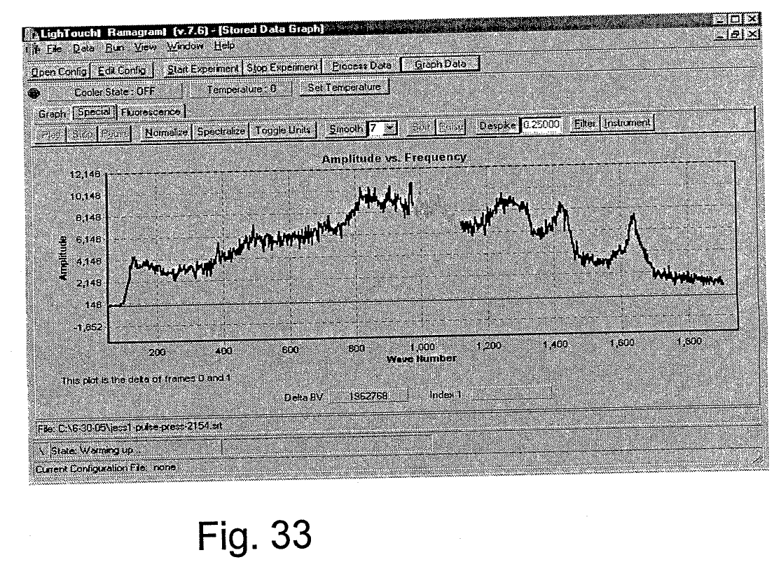

The ability to observe the pulse during the pressed stage also varied with the applied force during that stage. For low enough applied force as in the un-pressed stage, the pulses are easily observed. As can be observed in FIG. 32, as the target force is increased for the pressed state the pulses, i.e. either the actual force at the skin surface (shown) or the integral of the total emission above 800 cm⁻¹ (shown below in later figure), become less rounded and eventually have at least once sharp feature in their time profile. Increasing the target force more, the pulses become smaller in magnitude before eventually becoming unobservable. The force at which the pulses stop increases with the size of the fingertip being studied. Crudely, this “stopping” force increases with the area of contact between the fingertip and the spring steel surface or with the independently measured blood pressure of the subject.

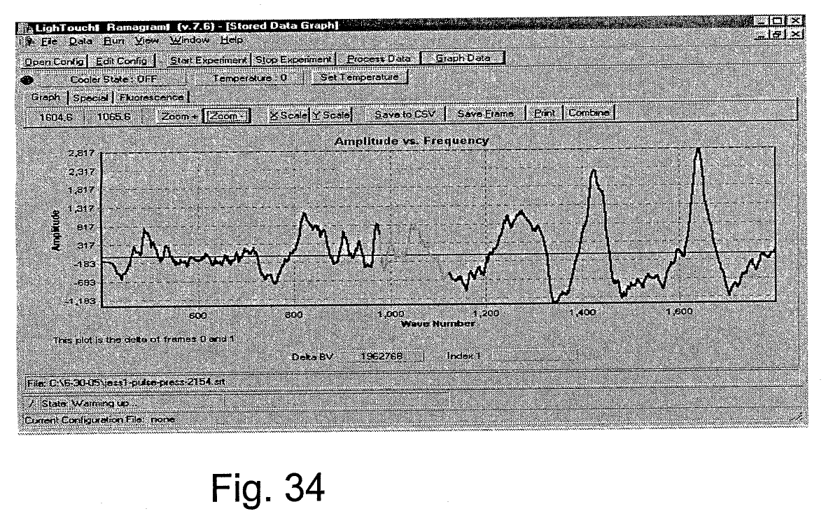

Within an un-pressed or pressed stage, by accumulating the CCD frames corresponding to the high emission in the blood volume versus time, i.e. the “tops” of the pulses, and subtracting the spectrum obtained by accumulating the frames corresponding to the low emission, i.e. the bottoms” of the pulses, pulse modulated spectra can be observed. FIGS. 33 and 34 are typical for a spectrum obtained during a pressed stage with intermediate high pressure target. This corresponds to a total accumulation time of 43 seconds and is shown both raw, FIG. 33 and background subtracted as in FIG. 34. It should be pointed out that we have observed difference spectra with higher and lower signal to noise ratio on the same timescale. Generally, spectra obtained by tissue modulation, i.e. subtracting accumulated pressed and un-pressed stages, have a very similar appearance to the pulse modulated spectra obtained from the same pressed data using pulse modulation, i.e. when pulses are observed. Pulse modulated spectra obtained using the un-pressed stage always have much greater fluorescence below 800 cm⁻¹ Raman shift than do spectra collected during the pressed stage. The resemblance between these spectra and earlier tissue modulated spectra published (Chaiken et al., Proc. SPIE, Vol. 3907, 89-97, 2000) is striking.

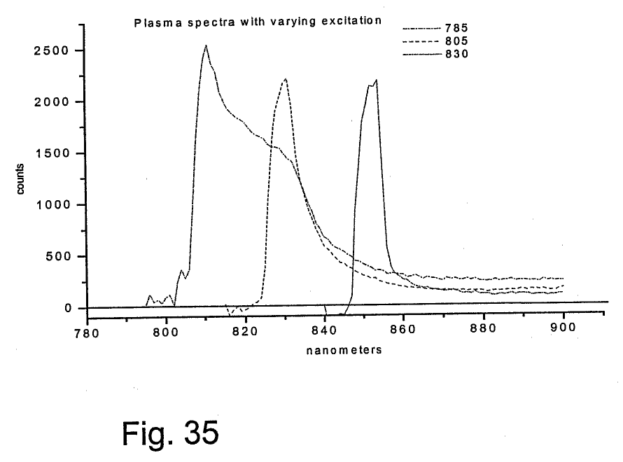

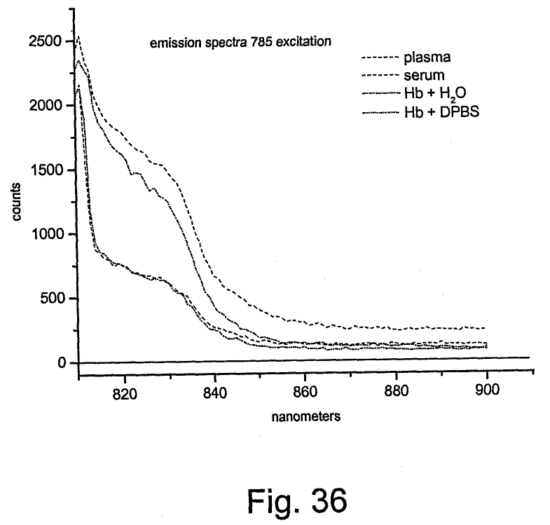

For purposes of clarifying the nature of the spectra collected in vivo, fluorescence spectra of blood sampled from a large vessel were obtained and are shown in FIGS. 35 and 36. Interestingly, in spite of the very large amount of published in vitro spectra of hemoglobin and porphyrins in the visible and UV spectral regions, we found no NIR excited fluorescence spectra. Based on observed visible emission spectra, it was neatly impossible to obtain plasma and serum samples that were not contaminated with hemoglobin. For comparison, small, ≈10¹-10² Liter samples of the hematocrit isolated from either the serum or plasma tubes were added to pure sterile, filtered deionized water and phosphate buffeted saline. The water and buffer samples had no emission whatsoever by themselves regardless of excitation wavelength. However, all 785 and 805 nm excited spectra of any of the blood components had an emission feature peaked near 820 nm. This feature was observed whether the excitation and emission collection were executed continuously in time or whether the excitation and emission collection were carried out without temporal overlap, i.e. 20 μsec excitation window followed by 1 msec emission window delayed by 20 μsec after end of excitation. On the other hand, fresh samples of serum and to a lesser degree plasma, under only continuous excitation and emission detection, occasionally showed very low level emission extending to long wavelengths than out limit with the current instrumentation.

The variation in the spectra with changing pressed target is reminiscent of a type of variation we have observed many times. For example, the data used in the earlier small clinical study clearly showed this effect. This emission was excited using 785 nm with the same test subject attempting to be uniform and consistent in the application of the same force for each stage every time. Nevertheless, at least partly due to the fact that these spectra were obtained without any teal time force feedback, there is a depletion region that extends to larger Raman shifts compared to 805 nm excitation. Comparison of the position of this depletion region and that for 805 nm excited spectra with the fluorescence spectra obtained from in vitro blood fractions strongly suggests that the depletion corresponds to less emission from hemoglobin for the given modulation conditions.

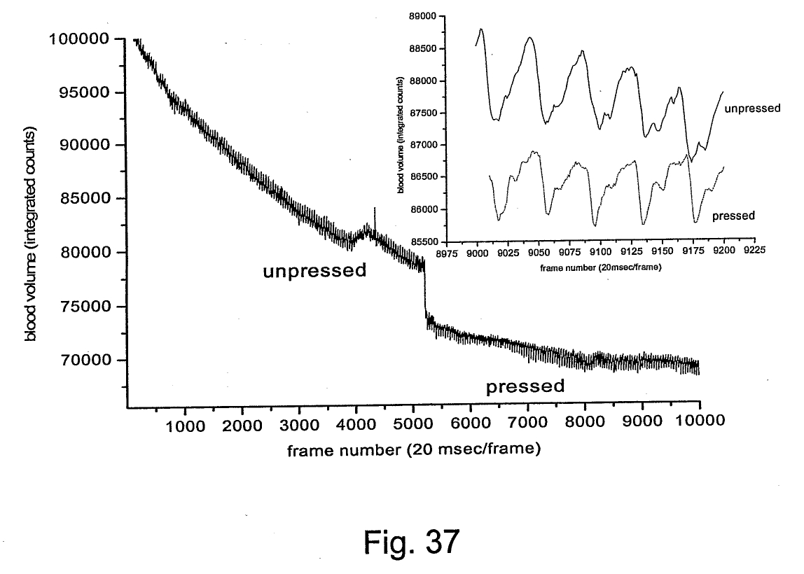

Independently, at shown in FIG. 37, the effect of the increasing pressure on the temporal appearance of the pulse also shows that the applied force is affecting the flow of the blood through the capillary system. This inference is further reinforced by the dependence of the pulse stopping force on the size of the contact region of the finger segment with the tissue modulating surface and the subjects blood pressure. The applied force divided by the contact area obtains the pressure against which the heart must push to obtain flow. This ratio will vary with changing finger size and diastolic and systolic blood pressure in a manner identical to our observations. For example, the applied force required to stop either the optically observed or the mechanically observed pulses, i.e. FIG. 32, increases with finger size. Note that if the fluorescence per unit volume of the plasma compared to the hematocrit were the same, then varying the proportions of each would not change the shaped of the optically detected pulse. Since we know from the in vitro measurements that the fluorescence pet unit volume of the two components are not equal, and although we do not have the capability to synchronize these measurements with an independent electrocardiogram signal, we can still speculate on how the flow is changed based on the temporal pressure profile.

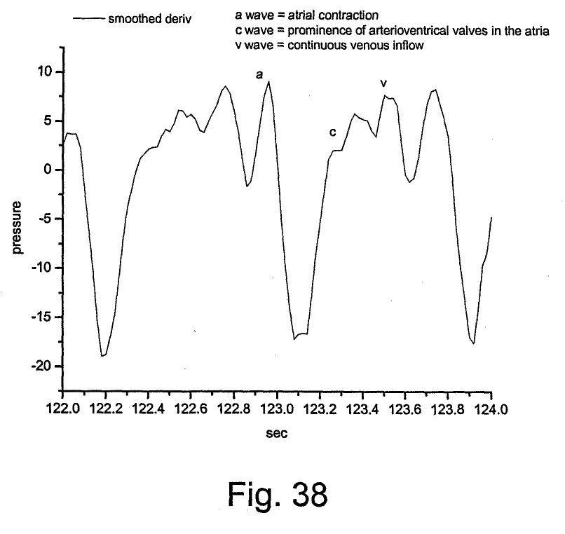

We can differentiate the observed pulse shapes, i.e. integrated emission intensity versus time, with respect to time to obtain the rate of change of emission as a function of time as shown in FIG. 38. This rate of change is the blood velocity that in turn is determined by the transient pressure via Poiseville's equation. The pressure profile produced in this way can be compared directly with reference pressure waves* corresponding to the pulmonary capillaries under wedge pressure. Although these observations correspond to very different parts of the anatomy, the only significant difference is that under “wedge pressure” the pulmonary capillaries release pressure between pulses due to the ability of the blood to flow around the obstruction presented by the catheter. In the present case the pressure drop is not nearly as pronounced due to the fact that the test subject does not release the applied force between pulses. * Geigy Tables, Page 18, Volume 5, Editor C. Lentner, 8^{th} Edition CIBA-GEIGY, Basel (1990)

These observations are consistent with a concept of the tissue modulation process whereby, at the lowest applied pressure, the blood flow is mostly unimpeded and we have a hematocrit change with an un-pressed-pressed cycle that reflects the Raman and fluorescence modulation corresponding to the largest available hematocrit. With increasing applied pressure, the capillaries begin to be occluded with increasing pressed target force. Depending on the pressed target pressure, and the test subject's systolic and diastolic blood pressure, the capillaries may or may not become completely blocked. Depending on the choice of excitation wavelength, the probed part of the vasculature may be relatively close to the surface, i.e. 785 nm, or it may be deeper into the capillary bed, i.e. 830 nm. The deeper the probing, the larger the normal diameter of the blood vessels probed. Plasma skimming is most important for vessels with inner diameters in the range of 20 μm<d<160 μm. These observations provide a basis for describing the effects we have observed.

The in vitro fluorescence spectra show that the NIR emission related to hemoglobin peaks at 820 nm regardless of excitation wavelength. The low Raman shift emission decreases because less of the chemical entitie(s) that are associated with these spectral features are moved or “modulated” by the applied forces. This is exactly as would be expected if the erythrocytes were being occluded before the motion of the plasma ceases. Thus, as observed, there should be less apparent low Raman shift emission, i.e. fluorescence, as the pressed target force is increased until the pulse is completely blocked. As observed, there should also be less Raman features associated with heme, globin and phospholipids as the target force is increased.

Note that if a Raman feature and a fluorescence feature are the only two contributions to the total emission at a particular emission wavelength, then the normalized emission at that wavelength, i.e. the ratio of the Raman emission to the fluorescence emission at that wavelength, should be nearly constant regardless of concentration, as observed. The fluid that is passing the excitation/observation zone in the capillary bed and that can monitored by the integrated total emission is plasma. There is a “bulge” of plasma that enters the observation zone and leaves with each heart pulse. The effective hematocrit can be adjusted by varying the pressed target force. The force needed to choose a particular hematocrit will vary from individual to individual since the pressure is the critical parameter and the contact area will change with different sized fingers. Either the pulse modulated or tissue modulated will yield comparable spectra because the same considerations apply to the nature of what is modulated. Note that this picture must be applied in both directions in that erythrocytes cannot get out of the excitation zone any more than they can newly enter. Thus the only material that is responsible for the changing spectra is plasma.

The spectroscopy we observe is on a material which is essentially trapped in the excitation/observation zone once the occlusion has occurred. We refer to this fluid as a “skim pool” since it is formed by the action of “plasma skimming” on the members of the vasculature whose internal diameters have been adjusted by external pressure to restrict the motion of erythrocytes while allowing the plasma to pass. We call either the tissue or pulse modulated spectra “skimmed” depending on the degree of fluorescence observed at low Raman shift. Since we understand the source of this effect we have extended our earlier approach to blood volume measurement to make this type of measurement more precise with respect to quantification of analytes. Nevertheless, we have presented the first Raman spectra of human blood plasma noninvasive, in vivo in this paper. These results are completely consistent with our earlier spectra of human capillary blood noninvasive, in vivo.

The importance of having spectra that demonstrably originate with blood cannot be overstated. For example, skin glucose measurements cannot be used in place of blood glucose measurements because skin glucose concentrations lag behind changes that occur in the blood by about 15-40 minutes depending on the individual and circumstances. This time lag could prove fatal for a person trying to avoid or at least ameliorate a hypoglycemic event. Since the existing quality of care involves blood measurements, and the lack of predictability of blood glucose measurements based on prior measurements, it seems important that for integration with insulin pumps, blood based measurements will be essential. Finally, we suspect that we have discovered a general effect that will be important in applying Raman spectroscopy to all tissues that have significant vasculature.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

What is claimed is:

1. An apparatus for noninvasive spectroscopic measurement of an analyte in a subject comprising:
(a) an ergonomically shaped grip that substantially conforms to a subject's hand;
(b) a surface for placement of at least one of the subject's fingertips upon grasping the grip; and
(c) an optically transparent aperture disposed within the surface.

2. The apparatus of claim 1, further comprising a modification to the surface adjacent to the aperture, wherein the modification is detectable via the tactile sense of the subject.

3. The apparatus of claim 2, wherein the modification comprises at least one raised nub, bump and/or ridge on the surface of the apparatus.

4. The apparatus of claim 1, further comprising one or more additional apertures.

5. The apparatus of claim 1, wherein the ergonomically shaped grip comprises two or more ridges that define recesses, whereby the recesses conform to the subject's fingers upon grasping the grip.

6. The apparatus of claim 1, wherein the grip is shaped to facilitate placement of the volar side of the subject's thumb tip on a portion of the grip that opposes the surface for placement of the subject's fingertips, whereby the subject moves the fingertips and opposing thumb tip towards each other upon grasping the grip.

7. The apparatus of claim 1, further comprising a sensor in the surface of the grip adjacent to the aperture, wherein the sensor detects proper positioning of a fingertip over the aperture.

8. The apparatus of claim 7, wherein the sensor comprises an electrode array.

9. The apparatus of claim 8, wherein the electrode array comprises a plurality of electrodes, and each electrode detects 60 Hz electrical activity.

10. The apparatus of claim 7, wherein the sensor detects pressure applied by a fingertip.

11. The apparatus of claim 7, wherein the sensor detects moisture.

12. The apparatus of claim 11, wherein moisture is detected via resistance, capacitance or impedance between the electrodes.

13. The apparatus of claim 1, further comprising a feedback loop that transmits a detectable signal that corresponds to information detected by the sensor.

14. The apparatus of claim 13, wherein the detectable signal is transmitted to a processor.

15. The apparatus of claim 13, wherein the detectable signal is transmitted to the subject.

16. The apparatus of claim 1, further comprising means for measuring temperature of the surface.

17. The apparatus of claim 1, further comprising means for altering the temperature of the surface.

18. The apparatus of claim 1, further comprising a spectroscopic measurement system that directs light through the aperture toward the subject's fingertip and detects spectra emitted from the subject's fingertip through the aperture.

19. A method for noninvasive spectroscopic measurement of an analyte in a subject comprising:
(a) contacting the subject's hand with an apparatus of claim 1;
(b) positioning a fingertip of the subject over the aperture;
(c) directing light through the aperture toward the subject's fingertip; and
(d) collecting and measuring spectra emitted from the subject's fingertip through the aperture, wherein the spectra correspond to the analyte to be measured.

20. The method of claim 19, further comprising:
(e) monitoring the pressure applied by the fingertip positioned over the aperture; and
(f) providing concurrent feedback to the subject regarding the pressure applied.

21. The method of claim 20, wherein the feedback directs the subject to maintain application of a predetermined force.

22. The method of claim 20, wherein the feedback directs the subject to maintain application of a predetermined force per unit area of the volar side of the fingertip.

23. The method of claim 19, further comprising:
(e) monitoring the position of the fingertip positioned over the aperture; and
(f) providing concurrent feedback to the subject regarding the position of the fingertip.

24. The method of claim 19, further comprising:
(e) monitoring the electrical resistance of the fingertip positioned over the aperture; and
(f) providing concurrent feedback to the subject regarding the moisture of the fingertip.

25. The method of claim 24, further comprising rejecting a spectral measurement if the resistance of the fingertip does not fall within a predetermined range.
